SEQUENCE LISTING (10 sequences)

SEQ ID NO: 1 - protein, 259 aa, Artificial
  Nanobody sequence or fragment thereof
EVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSSAAAEVQLVESGGGLVQPGGSLRLSCAASGRTFSYNPMGWFRQAPGKGRELVAAISRTGGSTYYPDSVEGRFTISRDNAKRMVYLQMNSLRAEDTAVYYCAAAGVRAEDGRVRTLPSEYTFWGQGTQVTVSS

SEQ ID NO: 2 - protein, 115 aa, Artificial [sequence omitted]

SEQ ID NO: 3 - protein, 387 aa, Artificial
  Nanobody sequence or fragment thereof
EVQLVESGGGLVQPGGSLRLSCAASGFTFSDYDIGWFRQAPGKGREGVSGISSSDGNTYYADSVKGRFTISRDNAKNTLYLQMNSLRPEDTAVYYCAAEPPDSSWYLDGSPEFFKYWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTFSDYDIGWFRQAPGKGREGVSGISSSDGNTYYADSVKGRFTISRDNAKNTLYLQMNSLRPEDTAVYYCAAEPPDSSWYLDGSPEFFKYWGQGTLVTVSS

SEQ ID NO: 4 - protein, 712 aa, Artificial
  Nanobody sequence or fragment thereof
EVQLVESGGGLVQPGGSLRLSCAASGFTFSDYDIGWFRQAPGKGREGVSGISSSDGNTYYADSVKGRFTISRDNAKNTLYLQMNSLRPEDTAVYYCAAEPPDSSWYLDGSPEFFKYWGQGTLVTVSSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 5 - protein, 251 aa, Artificial
  Nanobody sequence or fragment thereof
EVQLVESGGGLVQPGGSLRLSCAASGFTFSDYDIGWFRQAPGKGREGVSGISSSDGNTYYADSVKGRFTISRDNAKNTLYLQMNSLRPEDTAVYYCAAEPPDSSWYLDGSPEFFKYWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 6 - protein, 115 aa, Artificial [sequence omitted]

SEQ ID NO: 7 - protein, 214 aa, Artificial
  Human fragment of A1 vWF with tag
DISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTHHHHHH

SEQ ID NO: 8 - protein, 17 aa, Artificial
  Nanobody sequence or fragment thereof
GGSLSRSSQGTLVTVSS

SEQ ID NO: 9 - protein, 115 aa, Artificial [sequence omitted]

SEQ ID NO: 10 - protein, 115 aa, Artificial [sequence omitted]

(19) United States
(12) Patent Application Publication
(10) Pub. No.: US 2011/0091462 A1
(43) Pub. Date: Apr. 21, 2011

(54) NOVEL ANTIGEN BINDING DIMER-COMPLEXES, METHODS OF MAKING AND USES THEREOF

(75) Inventors: Peter Casteels, Erpe-Mere (BE); Marc Jozef Lauwereys, Haaltret (BE); Patrick Stanssens, Nazareth (BE); Christine Labeur, Brugge (BE); Carlo Boutton, Wielsbeke (BE); Ann Brigé, Ertvelde (BE); Hendricus Renerus Jacobus M Hoogenboom, Maastricht (NL); Els Anna Alice Beirnaert, Bellem (BE)

(73) Assignee: ABLYNX N.V., ZWIJNAARDE (BE)

(21) Appl. No.: 12/920,862

(22) PCT Filed: Mar. 5, 2009
(86) PCT No.: PCT/EP09/52629

§ 371 Date: Jan. 5, 2011

Related U.S. Application Data
(60) Provisional application No. 61/033,902, filed on Mar. 5, 2008.

(51) Int. Cl. C07K 16/00; A61K 39/395

(52) U.S. Cl. 424/135.1; 530/387.3

(57) ABSTRACT

In a broad aspect the present invention generally relates to novel dimer-complexes (herein called “non-fused-dimers” or NFDs) comprising single variable domains, methods of making these complexes and uses thereof. These non-covalently bound dimer-complexes consist of two identical monomers that each comprises of one or more single variable domains (homodimers) or of two different monomers that each comprises on or more single variable domains (heterodimers). The subject NFDs have typically altered e.g. improved binding characteristics over their monomeric counterpart. The NFDs of the invention may further be engineered through linkage by a flexible peptide or cysteines in order to improve the stability. This invention also describes conditions under which such NFDs are formed and conditions under which the formation of such dimers can be avoided.

In a broad aspect the present invention generally relates to novel dimer-complexes (herein called “non-fused-dimers” or NFDs) comprising single variable domains such as e.g. Nanobodies, methods of making these complexes and uses thereof. These non-covalently bound dimer-complexes consist of two identical monomers that each comprises of one or more single variable domains (homodimers) or of two different monomers that each comprises on or more single variable domains (heterodimers). The subject NFDs have typically altered e.g. improved or decreased binding characteristics over their monomeric counterpart. The NFDs of the invention may further be engineered through linkage by a flexible peptide or cysteines in order to improve the stability. This invention also describes conditions under which such NFDs are formed and conditions under which the formation of such dimers can be avoided. E.g. the present invention also provides methods for suppressing NFDs such as the dimerization of (human serum) albumin-binding Nanobodies by adding to a formulation one or more excipients that increase the melting temperature of the singe variable domain such as e.g. mannitol or other polyols to a liquid formulation.

### BACKGROUND OF THE INVENTION

The antigen binding sites of conventional antibodies are formed primarily by the hypervariable loops from both the heavy and the light chain variable domains. Functional antigen binding sites can however also be formed by heavy chain variable domains (VH) alone. In vivo, such binding sites have evolved in camels and camelids as part of antibodies, which consist only of two heavy chains and lack light chains. Furthermore, analysis of the differences in amino acid sequence between the VHs of these camel heavy chain-only antibodies (also referred to as VHH) and VH domains from conventional human antibodies helped to design altered human VH domains (Lutz Riechmann and Serge Muyldermans, J. of Immunological Methods, Vol. 231, Issues 1 to 2, 1999, 25-38). Similarly, it has been shown that by mutation studies of the interface residues as well as of the CDR3 on the VH of the anti-Her2 antibody 4D5 in parallel with the anti-hCG VHH H14, some mutations were found to promote autonomous VH domain behaviour (i.e. beneficial solubility and reversible refolding) (Barthelemy P A et al., 2008, J. of Biol. Chemistry, Vol 283, No 6, pp 3639-3654).

It was also found that increasing the hydrophilicity of the former light chain interface by replacing exposed hydrophobic residues by more hydrophilic residues improves the autonomous VH domain behaviour. These engineered VHs were shown to be predominantly monomeric at high concentration, however low quantities of dimers and other aggregates of said engineered VHs were also found that presumably form relative weak interaction similar to those described in the art for VL-VH pair interactions. Similarly, a camelized VH, called cVH-E2, is claimed to form dimers in solution in a concentration dependent manner i.e. at concentrations above 7 mg/ml (but note that data has not been shown in study; Dottorini et al. Biochemistry, 2004, 43, 622-628). Below this concentration, the dimer likely dissociates into monomers and it remains unclear whether these dimers were active (i.e. binding antigen). Furthermore, it has recently been reported that a truncated Llama derived VHH (the first seven amino acids are cleaved off) with a very short CDR3 (only 6 residues) called VHH-R9 forms a domain swapped dimer in the crystal structure. Since VHH-R9 has been shown to be functional in solution (low Kd against hapten) and to consist of a monomer only, it is likely that dimerization occurred during the very slow crystallization process (4 to 5 weeks) and that elements such as N-terminal cleavage, high concentration conditions and short CDR3 could lead or contribute to the “condensation” phenomena (see in particular also conclusion part of Spinelli et al. FEBS Letter 564, 2004, 35-40). Sepulveda et al. (J. Mol. Biol. (2003) 333, 355-365) has found that spontaneous formation of VH dimers (VHD) is in many cases permissive, producing molecules with antigen binding specificity. However, based on the reported spontaneous formation (versus the dimers formed by PIA reported herein) and the lack of stability data on the non-fused dimers, it is likely that these are weakly interacting dimers similar to the ones described by Barthelemy (supra). Taken together, the literature describes the formation of dimers of single variable domains and fragments thereof that a) are interacting primarily on relatively weak hydrophobic interaction (which are e.g. depending on the concentration, reversible), and/or b) occur in another occasion only in the crystallisation process (e.g. as a result of crystal packing forces). Moreover, it has been described that these dimers were not binding antigens anymore (as in Spinelli (supra)) or it is unclear whether these dimers were binding dimers (as in Dottorini (supra) and Barthelemy (supra)).

### DESCRIPTION OF THE INVENTION

It has now surprisingly been found that stable dimer-complexes can be generated in solution for polypeptides comprising at least one single variable VHH domain, preferably for polypeptides comprising single variable VHH domain that form dimers using the methods described herein (i.e. process-induced association, introduction of CDR3/framework region 4 destabilizing residues and/or storage at high temperature and high concentration), more preferably for polypeptides comprising at least one single variable VHH domain with sequences SEQ ID NO: 1 to 6 and/or variants thereof, e.g. single variable VHH domain with sequences that are 70% and more identical to SEQ ID NO: 1 to 6. Some of these stable dimer-complexes (also herein referred to as non-fused-dimers or NFDs; non-fused-dimer or NFD) can retain binding functionality to at least 50% or can even have increased binding affinity compared to their monomeric building blocks, others have decreased or no binding functionality anymore. These NFDs are much more stable compared to the ‘transient’ concentration-dependent dimers described e.g. in Barthelemy (supra) and are once formed stable in a wide range of concentrations. These NFDs may be formed by swapping framework 4 region between the monomeric building blocks whereby both said monomeric building blocks interlock (see experimental part of the crystal structure of polypeptide B NFD). These dimers are typically formed upon process-induced association (PIA) using methods described herein and/or storage at relative high temperature over weeks (such as e.g. 37° C. over 4 weeks) and high concentration (such as e.g. higher than 50 mg/ml, e.g. 65 mg/ml). The invention also teaches how to avoid the formation of said dimer-complexes in i) e.g. an up-scaled production or purification process of said polypeptides comprising single variable domain(s) under non-stress condition (i.e. condition that do not favour unfolding of immunoglobulins), ii) by an adequate formulation with excipients increasing the melting temperature of the single variable domain(s), e.g. by having mannitol in the formulation and/or iii) by increasing the stability of the CDR3 and/or framework 4 region conformation

### DEFINITIONS

a) Unless indicated or defined otherwise, all terms used have their usual meaning in the art, which will be clear to the skilled person. Reference is for example made to the standard handbooks, such as Sambrook et al, “Molecular Cloning: A Laboratory Manual” (2nd. Ed.), Vols. 1-3, Cold Spring Harbor Laboratory Press (1989); F. Ausubel et al, eds., “Current protocols in molecular biology”, Green Publishing and Wiley Interscience, New York (1987); Lewin, “Genes II”, John Wiley & Sons, New York, N.Y., (1985); Old et al., “Principles of Gene Manipulation: An Introduction to Genetic Engineering”, 2nd edition, University of California Press, Berkeley, Calif. (1981); Roitt et al., “Immunology” (6th. Ed.). Mosby/Elsevier, Edinburgh (2001); Roitt et al., Roitt's Essential Immunology, 10^{th} Ed. Blackwell. Publishing, UK (2001); and Janeway et al., “Immunobiology” (6th Ed.), Garland Science Publishing/Churchill Livingstone, N.Y. (2005), as well as to the general background art cited herein;
b) Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein; as well as to for example the following reviews Presta, Adv. Drug Deliv. Rev. 2006, 58 (5-6): 640-56; Levin and Weiss, MeI. Biosyst. 2006, 2(1): 49-57; Irving et al., J. Immunol. Methods, 2001, 248(1-2), 31-45; Schmitz et al., Placenta, 2000, 21 Suppl. A. S106-12, Gonzales et al., Tumour Biol., 2005, 26(1), 31-43, which describe techniques for protein engineering, such as affinity maturation and other techniques for improving the specificity and other desired properties of proteins such as immunoglobulins.
c) Amino acid residues will be indicated according to the standard three-letter or one-letter amino acid code, as mentioned in Table A-2;

TABLE A-2
one-letter and three-letter ammo acid code

| | | | |
|---|---|---|---|
| Nonpolar, | Alanine | Ala | A |
| uncharged | Valine | Val | V |
| (at pH 6.0- | Leucine | Leu | L |
| 7.0)⁽³⁾ | Isoleucine | Ile | I |
| | Phenylalanine | Phe | F |
| | Methionine⁽¹⁾ | Met | M |
| | Tryptophan | Trp | W |
| | Proline | Pro | P |
| Polar, | Glycine⁽²⁾ | Gly | G |
| uncharged | Serine | Ser | S |
| (at pH 6.0-7.0) | Threonine | Thr | T |
| | Cysteine | Cys | C |
| | Asparagine | Asn | N |
| | Glutamine | Gln | Q |
| | Tyrosine | Tyr | Y |
| Polar, | Lysine | Lys | K |
| charged | Arginine | Arg | R |
| (at pH 6.0-7.0) | Histidine⁽⁴⁾ | His | H |
| | Aspartate | Asp | D |
| | Glutamate | Glu | E |

Notes:
⁽¹⁾Sometimes also considered to be a polar uncharged amino acid.
⁽²⁾Sometimes also considered to be a nonpolar uncharged amino acid.
⁽³⁾As will be clear to the skilled person, the fact that an amino acid residue is referred to in this Table as being either charged or uncharged at pH 6.0 to 7.0 does not reflect in any way on the charge said amino acid residue may have at a pH lower than 6.0 and/or at a pH higher than 7.0; the amino acid residues mentioned in the Table can be either charged and/or uncharged at such a higher or lower pH, as will be clear to the skilled person.
⁽⁴⁾As is known in the art, the charge of a His residue is greatly dependant upon even small shifts in pH, but a His residu can generally be considered essentially uncharged at a pH of about 6.5.

d) For the purposes of comparing two or more nucleotide sequences, the percentage of “sequence identity” between a first nucleotide sequence and a second nucleotide sequence may be calculated by dividing [the number of nucleotides in the first nucleotide sequence that are identical to the nucleotides at the corresponding positions in the second nucleotide sequence] by [the total number of nucleotides in the first nucleotide sequence] and multiplying by [100%], in which each deletion, insertion, substitution or addition of a nucleotide in the second nucleotide sequence—compared to the first nucleotide sequence—is considered as a difference at a single nucleotide (position).
   Alternatively, the degree of sequence identity between two or more nucleotide sequences may be calculated using a known computer algorithm for sequence alignment such as NCBI Blast v2.0, using standard settings.
   Some other techniques, computer algorithms and settings for determining the degree of sequence identity are for example described in WO 04/037999, EP 0 967 284, EP 1 085 089, WO 00/55318, WO 00/78972, WO 98/49185 and GB 2 357 768-A.
   Usually, for the purpose of determining the percentage of “sequence identity” between two nucleotide sequences in accordance with the calculation method outlined hereinabove, the nucleotide sequence with the greatest number of nucleotides will be taken as the “first” nucleotide sequence, and the other nucleotide sequence will be taken as the “second” nucleotide sequence;
e) For the purposes of comparing two or more amino acid sequences, the percentage of “sequence identity” between a first amino acid sequence and a second amino acid sequence (also referred to herein as “amino acid identity”) may be calculated by dividing [the number of amino acid residues in the first amino acid sequence that are identical to the amino acid residues at the corresponding positions in the second amino acid sequence] by [the total number of amino acid residues in the first amino acid sequence] and multiplying by [100%], in which each deletion, insertion, substitution or addition of an amino acid residue in the second amino acid sequence—compared to the first amino acid sequence—is considered as a difference at a single amino acid residue (position), i.e. as an “amino acid difference” as defined herein.
   Alternatively, the degree of sequence identity between two amino acid sequences may be calculated, using a known computer algorithm, such as those mentioned above for determining the degree of sequence identity for nucleotide sequences, again using standard settings.
   Usually, for the purpose of determining the percentage of “sequence identity” between two amino acid sequences in accordance with the calculation method outlined hereinabove, the amino acid sequence with the greatest number of amino acid residues will be taken as the “first” amino acid sequence, and the other amino acid sequence will be taken as the “second” amino acid sequence.
   Also, in determining the degree of sequence identity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Such conservative amino acid substitutions are well known in the art, for example from WO 04/037999, GB-A-3 357 768, WO 98/49185, WO 00/46383 and WO 01/09300; and (preferred) types and/or combinations of such substitutions may be selected on the basis of the pertinent teachings from WO 04/037999 as well as WO 98/49185 and from the further references cited therein.
   Such conservative substitutions preferably are substitutions in which one amino acid within the following groups (a)-(e) is substituted by another amino acid residue within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His. Arg and Lys; (d) large aliphatic, nonpolar residues: Met, Leu, Ile, Val and Cys; and (e) aromatic residues: Phe, Tyr and Trp.
   Particularly preferred conservative substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile: Phe into Met, into Leu or into Tyr; Ser into Thr; Thr into Ser; Trp into Tyr; Tyr into Trp; and/or Phe into Val, into Ile or into Leu.
   Any amino acid substitutions applied to the polypeptides described herein may also be based on the analysis of the frequencies of amino acid variations between homologous proteins of different species developed by Schulz et al., Principles of Protein Structure, Springer-Verlag, 1978, on the analyses of structure forming potentials developed by Chou and Fasman, Biochemistry 13: 211, 1974 and Adv. Enzymol., 47: 45-149, 1978, and on the analysis of hydrophobicity patterns in proteins developed by Eisenberg et al., Proc. Natl. Acad. Sci. USA 81: 140-144, 1984; Kyte & Doolittle; J. Molec. Biol. 157: 105-132, 1981, and Goldman et al., Ann. Rev. Biophys. Chem. 15: 321-353, 1986, all incorporated herein in their entirety by reference. Information on the primary, secondary and tertiary structure of Nanobodies is given in the description herein and in the general background art cited above. Also, for this purpose, the crystal structure of a V_{HH} domain from a llama is for example given by Desmyter et al., Nature Structural Biology, Vol. 3, 9, 803 (1996); Spinelli et al., Natural Structural Biology (1996); 3, 752-757; and Decanniere et al., Structure, Vol. 7, 4, 361 (1999). Further information about some of the amino acid residues that in conventional. V_{H} domains form the V_{H}/V_{L} interface and potential camelizing substitutions on these positions can be found in the prior art cited above.
f) Amino acid sequences and nucleic acid sequences are said to be “exactly the same” if they have 100% sequence identity (as defined herein) over their entire length;
g) When comparing two amino acid sequences, the term “amino acid difference” refers to an insertion, deletion or substitution of a single amino acid residue on a position of the first sequence, compared to the second sequence; it being understood that two amino acid sequences can contain one, two or more such amino acid differences;
h) When a nucleotide sequence or amino acid sequence is said to “comprise” another nucleotide sequence or amino acid sequence, respectively, or to “essentially consist of” another nucleotide sequence or amino acid sequence, this may mean that the latter nucleotide sequence or amino acid sequence has been incorporated into the first mentioned nucleotide sequence or amino acid sequence, respectively, but more usually this generally means that the first mentioned nucleotide sequence or amino acid sequence comprises within its sequence a stretch of nucleotides or amino acid residues, respectively, that has the same nucleotide sequence or amino acid sequence, respectively, as the latter sequence, irrespective of how the first mentioned sequence has actually been generated or obtained (which may for example be by any suitable method described herein). By means of a non-limiting example, when a Nanobody of the invention is said to comprise a CDR sequence, this may mean that said CDR sequence has been incorporated into the Nanobody of the invention, but more usually this generally means that the Nanobody of the invention contains within its sequence a stretch of amino acid residues with the same amino acid sequence as said CDR sequence, irrespective of how said Nanobody of the invention has been generated or obtained. It should also be noted that when the latter amino acid sequence has a specific biological or structural function, it preferably has essentially the same, a similar or an equivalent biological or structural function in the first mentioned amino acid sequence (in other words, the first mentioned amino acid sequence is preferably such that the latter sequence is capable of performing essentially the same, a similar or an equivalent biological or structural function). For example, when a Nanobody of the invention is said to comprise a CDR sequence or framework sequence, respectively, the CDR sequence and framework are preferably capable, in said Nanobody, of functioning as a CDR sequence or framework sequence, respectively. Also, when a nucleotide sequence is said to comprise another nucleotide sequence, the first mentioned nucleotide sequence is preferably such that, when it is expressed into an expression product (e.g. a polypeptide), the amino acid sequence encoded by the latter nucleotide sequence forms part of said expression product (in other words, that the latter nucleotide sequence is in the same reading frame as the first mentioned, larger nucleotide sequence).
i) A nucleic acid sequence or amino acid sequence is considered to be “(in) essentially isolated (form)”—for example, compared to its native biological source and/or the reaction medium or cultivation medium from which it has been obtained—when it has been separated from at least one other component with which it is usually associated in said source or medium, such as another nucleic acid, another protein/polypeptide, another biological component or macromolecule or at least one contaminant, impurity or minor component. In particular, a nucleic acid sequence or amino acid sequence is considered “essentially isolated” when it has been purified at least 2-fold, in particular at least 10-fold, more in particular at least 100-fold, and up to 1000-fold or more. A nucleic acid sequence or amino acid sequence that is “in essentially isolated form” is preferably essentially homogeneous, as determined using a suitable technique, such as a suitable chromatographical technique, such as polyacrylamide-gel electrophoresis;
j) The term “domain” as used herein generally refers to a globular region of an amino acid sequence (such as an antibody chain, and in particular to a globular region of a heavy chain antibody), or to a polypeptide that essentially consists of such a globular region. Usually, such a domain will comprise peptide loops (for example 3 or 4 peptide loops) stabilized, for example, as a sheet or by disulfide bonds. The term “binding domain” refers to such a domain that is directed against an antigenic determinant (as defined herein);
k) The term “antigenic determinant” refers to the epitope on the antigen recognized by the antigen-binding molecule (such as a Nanobody or a polypeptide of the invention) and more in particular by the antigen-binding site of said molecule. The terms “antigenic determinant” and “epitope” may also be used interchangeably herein.
l) An amino acid sequence (such as a Nanobody, an antibody, a polypeptide of the invention, or generally an antigen binding protein or polypeptide or a fragment thereof) that can (specifically) bind to, that has affinity for and/or that has specificity for a specific antigenic determinant, epitope, antigen or protein (or for at least one part, fragment or epitope thereof) is said to be “against” or “directed against” said antigenic determinant, epitope, antigen or protein.
m) The term “specificity” refers to the number of different types of antigens or antigenic determinants to which a particular antigen-binding molecule or antigen-binding protein (such as a Nanobody or a polypeptide of the invention) molecule can bind. The specificity of an antigen-binding protein can be determined based on affinity and/or avidity. The affinity, represented by the equilibrium constant for the dissociation of an antigen with an antigen-binding protein (K_{D}), is a measure for the binding strength between an antigenic determinant and an antigen-binding site on the antigen-binding protein: the lesser the value of the K_{D}, the stronger the binding strength between an antigenic determinant and the antigen-binding molecule (alternatively, the affinity can also be expressed as the affinity constant (K_{A}), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as a Nanobody or polypeptide of the invention) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the amino acid sequences. Nanobodies and/or polypeptides of the invention) will bind to their antigen with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles). Any K_{D} value greater than 10⁴ mol/liter (or any K_{A} value lower than 10⁴ M⁻¹) liters/mol is generally considered to indicate non-specific binding. Preferably, a monovalent immunoglobulin sequence of the invention will bind to the desired antigen with an affinity less than 500 nM, preferably less than 200 nM, more preferably less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (ETA) and sandwich competition assays, and the different variants thereof known per se in the art; as well as the other techniques mentioned herein.
   The dissociation constant may be the actual or apparent dissociation constant, as will be clear to the skilled person. Methods for determining the dissociation constant will be clear to the skilled person, and for example include the techniques mentioned herein. In this respect, it will also be clear that it may not be possible to measure dissociation constants of more then 10⁻⁴ moles/liter or 10⁻³ moles/liter (e.g. of 10⁻² moles/liter). Optionally, as will also be clear to the skilled person, the (actual or apparent) dissociation constant may be calculated on the basis of the (actual or apparent) association constant (K_{A}), by means of the relationship [K_{D}=1/K_{A}].
   The affinity denotes the strength or stability of a molecular interaction. The affinity is commonly given as by the K_{D}, or dissociation constant, which has units of mol/liter (or M). The affinity can also be expressed as an association constant. K_{A}, which equals 1/K_{D} and has units of (mol/liter)⁻¹ (or M⁻¹). In the present specification, the stability of the interaction between two molecules (such as an amino acid sequence, Nanobody or polypeptide of the invention and its intended target) will mainly be expressed in terms of the K_{D} value of their interaction; it being clear to the skilled person that in view of the relation K_{A}=1/K_{D}, specifying the strength of molecular interaction by its K_{D} value can also be used to calculate the corresponding K_{A} value. The K_{D}-value characterizes the strength of a molecular interaction also in a thermodynamic sense as it is related to the free energy (DG) of binding by the well known relation DG=RT·ln(K_{D}) (equivalently DG=−RT·ln(K_{A})), where R equals the gas constant. T equals the absolute temperature and ln denotes the natural logarithm.
   The K_{D} for biological interactions which are considered meaningful (e.g. specific) are typically in the range of 10⁻¹⁰M (0.1 nM) to 10⁻⁵M (10000 nM). The stronger an interaction is, the lower is its K_{D}.
   The K_{D} can also be expressed as the ratio of the dissociation rate constant of a complex, denoted as k_{off}, to the rate of its association, denoted kₒₙ (so that K_{D}=k_{off}/kₒₙ and K_{A}=kₒₙ/k_{off}). The off-rate k_{off} has units s⁻¹ (where s is the SI unit notation of second). The on-rate kₒₙ has units M⁻¹s⁻¹. The on-rate may vary between 10² M⁻¹s⁻¹ to about 10⁷ M⁻¹s⁻¹, approaching the diffusion-limited association rate constant for bimolecular interactions. The off-rate is related to the half-life of a given molecular interaction by the relation t_{1/2}=ln(2)/k_{off}. The off-rate may vary between 10⁻⁶ s⁻¹ (near irreversible complex with a t_{1/2} of multiple days) to 1 s⁻¹ (t_{1/2}=0.69 s).
   The affinity of a molecular interaction between two molecules can be measured via different techniques known per se, such as the well known surface plasmon resonance (SPR) biosensor technique (see for example Ober et al., Intern. Immunology, 13, 1551-1559, 2001) where one molecule is immobilized on the biosensor chip and the other molecule is passed over the immobilized molecule under flow conditions yielding kₒₙ, k_{off} measurements and hence K_{D} (or K_{A}) values. This can for example be performed using the well-known BIACORE instruments.
   It will also be clear to the skilled person that the measured K_{D} may correspond to the apparent K_{D} if the measuring process somehow influences the intrinsic binding affinity of the implied molecules for example by artefacts related to the coating on the biosensor of one molecule. Also, an apparent K_{D} may be measured if one molecule contains more than one recognition sites for the other molecule. In such situation the measured affinity may be affected by the avidity of the interaction by the two molecules.
   Another approach that may be used to assess affinity is the 2-step ELISA (Enzyme-Linked Immunosorbent Assay) procedure of Friguet et al. (J. Immunol. Methods, 77, 305-19, 1985). This method establishes a solution phase binding equilibrium measurement and avoids possible artefacts relating to adsorption of one of the molecules on a support such as plastic.
   However, the accurate measurement of K_{D} may be quite labor-intensive and as consequence, often apparent K_{D} values are determined to assess the binding strength of two molecules. It should be noted that as long all measurements are made in a consistent way (e.g. keeping the assay conditions unchanged) apparent K_{D} measurements can be used as an approximation of the true K_{D} and hence in the present document K_{D} and apparent K_{D} should be treated with equal importance or relevance. Finally, it should be noted that in many situations the experienced scientist may judge it to be convenient to determine the binding affinity relative to some reference molecule. For example, to assess the binding strength between molecules A and B, one may e.g. use a reference molecule C that is known to bind to B and that is suitably labelled with a fluorophore or chromophore group or other chemical moiety, such as biotin for easy detection in an ELISA or FACS (Fluorescent activated cell sorting) or other format (the fluorophore for fluorescence detection, the chromophore for light absorption detection, the biotin for streptavidin-mediated ELISA detection). Typically, the reference molecule C is kept at a fixed concentration and the concentration of A is varied for a given concentration or amount of B. As a result an IC₅₀ value is obtained corresponding to the concentration of A at which the signal measured for C in absence of A is halved. Provided K_{D ref}, the K_{D} of the reference molecule, is known, as well as the total concentration c_{ref} of the reference molecule, the apparent K_{D} for the interaction A-B can be obtained from following formula: K_{D}=IC₅₀/(1+c_{ref}/K_{D ref}). Note that if c_{ref}<<K_{D ref}, K_{D}≈IC₅₀. Provided the measurement of the IC₅₀ is performed in a consistent way (e.g. keeping c_{ref} fixed) for the binders that are compared, the strength or stability of a molecular interaction can be assessed by the IC₅₀ and this measurement is judged as equivalent to K_{D} or to apparent K_{D} throughout this text.
n) The half-life of an amino acid sequence, compound or polypeptide of the invention can generally be defined as the time taken for the serum concentration of the amino acid sequence, compound or polypeptide to be reduced by 50%, in vivo, for example due to degradation of the sequence or compound and/or clearance or sequestration of the sequence or compound by natural mechanisms. The in vivo half-life of an amino acid sequence, compound or polypeptide of the invention can be determined in any manner known per se, such as by pharmacokinetic analysis. Suitable techniques will be clear to the person skilled in the art, and may for example generally involve the steps of suitably administering to a warm-blooded animal (i.e. to a human or to another suitable mammal, such as a mouse, rabbit, rat, pig, dog or a primate, for example monkeys from the genus Macaca (such as, and in particular, cynomolgus monkeys (Macaca fascicularis) and/or rhesus monkeys (Macaca mulatta)) and baboon (Papio ursinus)) a suitable dose of the amino acid sequence, compound or polypeptide of the invention; collecting blood samples or other samples from said animal; determining the level or concentration of the amino acid sequence, compound or polypeptide of the invention in said blood sample; and calculating, from (a plot of) the data thus obtained, the time until the level or concentration of the amino acid sequence, compound or polypeptide of the invention has been reduced by 50% compared to the initial level upon dosing. Reference is for example made to the Experimental Part below, as well as to the standard handbooks, such as Kenneth, A et al: Chemical Stability of Pharmaceuticals: A Handbook for Pharmacists and Peters et al, Pharmacokinete analysis: A Practical Approach (1996). Reference is also made to “Pharmacokinetics”, M Gibaldi & D Perron, published by Marcel Dekker, 2nd Rev. edition (1982).
   As will also be clear to the skilled person (see for example pages 6 and 7 of WO 04/003019 and in the further references cited therein), the half-life can be expressed using parameters such as the t½-alpha, t½-beta and the area under the curve (AUC). In the present specification, an “increase in half-life” refers to an increase in any one of these parameters, such as any two of these parameters, or essentially all three these parameters. As used herein “increase in half-life” or “increased half-life” in particular refers to an increase in the t½-beta, either with or without an increase in the t½-alpha and/or the AUC or both.
o) In the context of the present invention, “modulating” or “to modulate” generally means either reducing or inhibiting the activity of, or alternatively increasing the activity of, a target or antigen, as measured using a suitable in vitro, cellular or in vivo assay. In particular, “modulating” or “to modulate” may mean either reducing or inhibiting the activity of, or alternatively increasing a (relevant or intended) biological activity of, a target or antigen, as measured using a suitable in vitro, cellular or in vivo assay (which will usually depend on the target or antigen involved), by at least 1%, preferably at least 5%, such as at least 10% or at least 25%, for example by at least 50%, at least 60%, at least 70%, at least 80%, or 90% or more, compared to activity of the target or antigen in the same assay under the same conditions but without the presence of the construct of the invention.
   As will be clear to the skilled person, “modulating” may also involve effecting a change (which may either be an increase or a decrease) in affinity, avidity, specificity and/or selectivity of a target or antigen for one or more of its ligands, binding partners, partners for association into a homomultimeric or heteromultimeric form, or substrates; and/or effecting a change (which may either be an increase or a decrease) in the sensitivity of the target or antigen for one or more conditions in the medium or surroundings in which the target or antigen is present (such as pH, ion strength, the presence of co-factors, etc.), compared to the same conditions but without the presence of the construct of the invention. As will be clear to the skilled person, this may again be determined in any suitable manner and/or using any suitable assay known per se, depending on the target or antigen involved.
   “Modulating” may also mean effecting a change (i.e. an activity as an agonist, as an antagonist or as a reverse agonist, respectively, depending on the target or antigen and the desired biological or physiological effect) with respect to one or more biological or physiological mechanisms, effects, responses, functions, pathways or activities in which the target or antigen (or in which its substrate(s), ligand(s) or pathway(s) are involved, such as its signalling pathway or metabolic pathway and their associated biological or physiological effects) is involved. Again, as will be clear to the skilled person, such an action as an agonist or an antagonist may be determined in any suitable manner and/or using any suitable (in vitro and usually cellular or in assay) assay known per se, depending on the target or antigen involved. In particular, an action as an agonist or antagonist may be such that an intended biological or physiological activity is increased or decreased, respectively, by at least 1%, preferably at least 5%, such as at least 10% or at least 25%, for example by at least 50%, at least 60%, at least 70%, at least 80%, or 90% or more, compared to the biological or physiological activity in the same assay under the same conditions but without the presence of the construct of the invention.
   Modulating may for example also involve allosteric modulation of the target or antigen; and/or reducing or inhibiting the binding of the target or antigen to one of its substrates or ligands and/or competing with a natural ligand, substrate for binding to the target or antigen. Modulating may also involve activating the target or antigen or the mechanism or pathway in which it is involved. Modulating may for example also involve effecting a change in respect of the folding or confirmation of the target or antigen, or in respect of the ability of the target or antigen to fold, to change its confirmation (for example, upon binding of a ligand), to associate with other (sub)units, or to disassociate. Modulating may for example also involve effecting a change in the ability of the target or antigen to transport other compounds or to serve as a channel for other compounds (such as ions).
   Modulating may be reversible or irreversible, but for pharmaceutical and pharmacological purposes will usually be in a reversible manner.
p) In respect of a target or antigen, the term “interaction site” on the target or antigen means a site, epitope, antigenic determinant, part, domain or stretch of amino acid residues on the target or antigen that is a site for binding to a ligand, receptor or other binding partner, a catalytic site, a cleavage site, a site for allosteric interaction, a site involved in multi-merization (such as homomerization or heterodimerization) of the target or antigen; or any other site, epitope, antigenic determinant, part, domain or stretch of amino acid residues on the target or antigen that is involved in a biological action or mechanism of the target or antigen. More generally, an “interaction site” can be any site, epitope, antigenic determinant, part, domain or stretch of amino acid residues on the target or antigen to which an amino acid sequence or polypeptide of the invention can bind such that the target or antigen (and/or any pathway, interaction, signalling, biological mechanism or biological effect in which the target or antigen is involved) is modulated (as defined herein).
q) An amino acid sequence or polypeptide is said to be “specific for” a first target or antigen compared to a second target or antigen when is binds to the first antigen with an affinity (as described above, and suitably expressed as a K_{D} value, K_{A} value. K_{off} rate and/or Kₒₙ rate) that is at least 10 times, such as at least 100 times, and preferably at least 1000 times, and up to 10,000 times or more better than the affinity with which said amino acid sequence or polypeptide binds to the second target or polypeptide. For example, the first antigen may bind to the target or antigen with a K_{D} value that is at least 10 times less, such as at least 100 times less, and preferably at least 1000 times less, such as 10,000 times less or even less than that, than the K_{D} with which said amino acid sequence or polypeptide binds to the second target or polypeptide. Preferably, when an amino acid sequence or polypeptide is “specific for” a first target or antigen compared to a second target or antigen, it is directed against (as defined herein) said first target or antigen, but not directed against said second target or antigen.
r) The terms “cross-block”, “cross-blocked” and “cross-blocking” are used interchangeably herein to mean the ability of an amino acid sequence or other binding agents (such as a polypeptide of the invention) to interfere with the binding of other amino acid sequences or binding agents of the invention to a given target. The extend to which an amino acid sequence or other binding agents of the invention is able to interfere with the binding of another to [target], and therefore whether it can be said to cross-block according to the invention, can be determined using competition binding assays. One particularly suitable quantitative assay uses a Biacore machine which can measure the extent of interactions using surface plasmon resonance technology. Another suitable quantitative cross-blocking assay uses an ELISA-based approach to measure competition between amino acid sequence or another binding agents in terms of their binding to the target.
   The following generally describes a suitable Biacore assay for determining whether an amino acid sequence or other binding agent cross-blocks or is capable of cross-blocking according to the invention. It will be appreciated that the assay can be used with any of the amino acid sequence or other binding agents described herein. The Biacore machine (for example the Biacore 3000) is operated in line with the manufacturer's recommendations. Thus in one cross-blocking assay, the target protein is coupled to a CM5 Biacore chip using standard amine coupling chemistry to generate a surface that is coated with the target. Typically 200-800 resonance units of the target would be coupled to the chip (an amount that gives easily measurable levels of binding but that is readily saturable by the concentrations of test reagent being used). Two test amino acid sequences (termed A* and B*) to be assessed for their ability to cross-block each other are mixed at a one to one molar ratio of binding sites in a suitable buffer to create the test mixture. When calculating the concentrations on a binding site basis the molecular weight of an amino acid sequence is assumed to be the total molecular weight of the amino acid sequence divided by the number of target binding sites on that amino acid sequence. The concentration of each amino acid sequence in the test mix should be high enough to readily saturate the binding sites for that amino acid sequence on the target molecules captured on the Biacore chip. The amino acid sequences in the mixture are at the same molar concentration (on a binding basis) and that concentration would typically be between 1.00 and 1.5 micromolar (on a binding site basis). Separate solutions containing A* alone and B* alone are also prepared. A* and B* in these solutions should be in the same buffer and at the same concentration as in the test mix. The test mixture is passed over the target-coated Biacore chip and the total amount of binding recorded. The chip is then treated in such a way as to remove the bound amino acid sequences without damaging the chip-bound target. Typically this is done by treating the chip with 30 mM HCl for 60 seconds. The solution of A* alone is then passed over the target-coated surface and the amount of binding recorded. The chip is again treated to remove all of the bound amino acid sequences without damaging the chip-bound target. The solution of B* alone is then passed over the target-coated surface and the amount of binding recorded. The maximum theoretical binding of the mixture of A* and B* is next calculated, and is the sum of the binding of each amino acid sequence when passed over the target surface alone. If the actual recorded binding of the mixture is less than this theoretical maximum then the two amino acid sequences are cross-blocking each other. Thus, in general, a cross-blocking amino acid sequence or other binding agent according to the invention is one which will bind to the target in the above Biacore cross-blocking assay such that during the assay and in the presence of a second amino acid sequence or other binding agent of the invention the recorded binding is between 80% and 0.1% (e.g. 80% to 4%) of the maximum theoretical binding, specifically between 75% and 0.1% (e.g. 75% to 4%) of the maximum theoretical binding, and more specifically between 70% and 0.1% (e.g. 70% to 4%) of maximum theoretical binding (as just defined above) of the two amino acid sequences or binding agents in combination. The Biacore assay described above is a primary assay used to determine if amino acid sequences or other binding agents cross-block each other according to the invention. On rare occasions particular amino acid sequences or other binding agents may not bind to target coupled via amine chemistry to a CM5 Biacore chip (this usually occurs when the relevant binding site on target is masked or destroyed by the coupling to the chip). In such cases cross-blocking can be determined using a tagged version of the target, for example a N-terminal His-tagged version (R & D Systems, Minneapolis. MN, USA; 2005 cat#1406-ST-025). In this particular format, an anti-His amino acid sequence would be coupled to the Biacore chip and then the His-tagged target would be passed over the surface of the chip and captured by the anti-H is amino acid sequence. The cross blocking analysis would be carried out essentially as described above, except that after each chip regeneration cycle, new His-tagged target would be loaded back onto the anti-His amino acid sequence coated surface. In addition to the example given using N-terminal His-tagged [target], C-terminal His-tagged target could alternatively be used. Furthermore, various other tags and tag binding protein combinations that are known in the art could be used for such a cross-blocking analysis (e.g. HA tag with anti-HA antibodies; FLAG tag with anti-FLAG antibodies; biotin tag with streptavidin).
   The following generally describes an ELISA assay for determining whether an amino acid sequence or other binding agent directed against a target cross-blocks or is capable of cross-blocking as defined herein. It will be appreciated that the assay can be used with any of the amino acid sequences (or other binding agents such as polypeptides of the invention) described herein. The general principal of the assay is to have an amino acid sequence or binding agent that is directed against the target coated onto the wells of an ELISA plate. An excess amount of a second, potentially cross-blocking, anti-target amino acid sequence is added in solution (i.e. not bound to the ELISA plate). A limited amount of the target is then added to the wells. The coated amino acid sequence and the amino acid sequence in solution compete for binding of the limited number of target molecules. The plate is washed to remove excess target that has not been bound by the coated amino acid sequence and to also remove the second, solution phase amino acid sequence as well as any complexes formed between the second, solution phase amino acid sequence and target. The amount of bound target is then measured using a reagent that is appropriate to detect the target. An amino acid sequence in solution that is able to cross-block the coated amino acid sequence will be able to cause a decrease in the number of target molecules that the coated amino acid sequence can bind relative to the number of target molecules that the coated amino acid sequence can bind in the absence of the second, solution phase, amino acid sequence. In the instance where the first amino acid sequence, e.g. an Ab-X, is chosen to be the immobilized amino acid sequence, it is coated onto the wells of the ELISA plate, after which the plates are blocked with a suitable blocking solution to minimize non-specific binding of reagents that are subsequently added. An excess amount of the second amino acid sequence, i.e. Ab-Y, is then added to the ELISA plate such that the moles of Ab-Y [target] binding sites per well are at least 10 fold higher than the moles of Ab-X [target] binding sites that were used, per well, during the coating of the ELISA plate. [target] is then added such that the moles of [target] added per well are at least 25-fold lower than the moles of Ab-X [target] binding sites that were used for coating each well. Following a suitable incubation period the ELISA plate is washed and a reagent for detecting the target is added to measure the amount of target specifically bound by the coated anti-[target] amino acid sequence (in this case Ab-X). The background signal for the assay is defined as the signal obtained in wells with the coated amino acid sequence (in this case Ab-X), second solution phase amino acid sequence (in this case Ab-Y), [target] buffer only (i.e. no target) and target detection reagents. The positive control signal for the assay is defined as the signal obtained in wells with the coated amino acid sequence (in this case Ab-X), second solution phase amino acid sequence buffer only (i.e. no second solution phase amino acid sequence), target and target detection reagents. The ELISA assay may be run in such a manner so as to have the positive control signal be at least 6 times the background signal. To avoid any artefacts (e.g. significantly different affinities between Ab-X and Ab-Y for [target]) resulting from the choice of which amino acid sequence to use as the coating amino acid sequence and which to use as the second (competitor) amino acid sequence, the cross-blocking assay may to be run in two formats: 1) format 1 is where Ab-X is the amino acid sequence that is coated onto the ELISA plate and Ab-Y is the competitor amino acid sequence that is in solution and 2) format 2 is where Ab-Y is the amino acid sequence that is coated onto the ELISA plate and Ab-X is the competitor amino acid sequence that is in solution. Ab-X and Ab-Y are defined as cross-blocking if, either in format 1 or in format 2, the solution phase anti-target amino acid sequence is able to cause a reduction of between 60% and 100%, specifically between 70% and 100%, and more specifically between 80% and 100%, of the target detection signal {i.e. the amount of target bound by the coated amino acid sequence) as compared to the target detection signal obtained in the absence of the solution phase anti-target amino acid sequence (i.e. the positive control wells).
s) As further described herein, the total number of amino acid residues in a Nanobody can be in the region of 110-120, is preferably 112-115, and is most preferably 113. It should however be noted that parts, fragments, analogs or derivatives (as further described herein) of a Nanobody are not particularly limited as to their length and/or size, as long as such parts, fragments, analogs or derivatives meet the further requirements outlined herein and are also preferably suitable for the purposes described herein;
t) The amino acid residues of a Nanobody are numbered according to the general numbering for V_{H} domains given by Kabat et al. (“Sequence of proteins of immunological interest”, US Public Health Services. NIH Bethesda, Md. Publication No. 91), as applied to V_{HH} domains from Camelids in the article of Riechmann and Muyldermans, J. Immunol. Methods 2000 Jun. 23; 240 (1-2): 185-195 (see for example FIG. 2 of this publication); or referred to herein. According to this numbering, FR1 of a Nanobody comprises the amino acid residues at positions 1-30, CDR1 of a Nanobody comprises the amino acid residues at positions 31-35, FR2 of a Nanobody comprises the amino acids at positions 36-49, CDR2 of a Nanobody comprises the amino acid residues at positions 50-65, FR3 of a Nanobody comprises the amino acid residues at positions 66-94, CDR3 of a Nanobody comprises the amino acid residues at positions 95-102, and FR4 of a Nanobody comprises the amino acid residues at positions 103-113. [In this respect, it should be noted that—as is well known in the art for V_{H} domains and for V_{HH} domains—the total number of amino acid residues in each of the CDR's may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence. Generally, however, it can be said that, according to the numbering of Kabat and irrespective of the number of amino acid residues in the CDR's, position 1 according to the Kabat numbering corresponds to the start of FR1 and vice versa, position 36 according to the Kabat numbering corresponds to the start of FR2 and vice versa, position 66 according to the Kabat numbering corresponds to the start of FR3 and vice versa, and position 103 according to the Kabat numbering corresponds to the start of FR4 and vice versa.]. Alternative methods for numbering the amino acid residues of V_{H} domains, which methods can also be applied in an analogous manner to V_{HH} domains from Camelids and to Nanobodies, are the method described by Chothia et al. (Nature 342, 877-883 (1989)), the so-called “AbM definition” and the so-called “contact definition”. However, in the present description, claims and figures, the numbering according to Kabat as applied to V_{HH} domains by Riechmann and Muyldermans will be followed, unless indicated otherwise;
u) By the term “Target Molecule” or “Target Molecules” or “target” is meant a protein with a biological function in an organism including bacteria and virus, preferably animal, more preferably mammal most preferred human, wherein said biological function may be involved in the initiation or progression or maintenance of a disease;
v) The single variable domains that are present in the constructs of the invention may be any variable domain that forms a single antigen binding unit. Generally, such single variable domains will be amino acid sequences that essentially consist of 4 framework regions (FR1 to FR4 respectively) and 3 complementarity determining regions (CDR1 to CDR3 respectively); or any suitable fragment of such an amino acid sequence (which will then usually contain at least some of the amino acid residues that form at least one of the CDR's, as further described herein). Such single variable domains and fragments are most preferably such that they comprise an immunoglobulin fold or are capable for forming, under suitable conditions, an immunoglobulin fold. As such, the single variable domain may for example comprise a light chain variable domain sequence (e.g. a V_{L}-sequence) or a suitable fragment thereof; or a heavy chain variable domain sequence (e.g. a V_{H}-sequence or V_{HH} sequence) or a suitable fragment thereof; as long as it is capable of forming a single antigen binding unit (i.e. a functional antigen binding unit that essentially consists of the single variable domain, such that the single antigen binding domain does not need to interact with another variable domain to form a functional antigen binding unit, as is for example the case for the variable domains that are present in for example conventional antibodies and ScFv fragments that need to interact with another variable domain—e.g. through a V_{H}/V_{L}, interaction—to form a functional antigen binding domain).
   For example, the single variable domain may be a domain antibody (or an amino acid sequence that is suitable for use as a domain antibody), a single domain antibody (or an amino acid sequence that is suitable for use as a single domain antibody), a “dAb” or dAb (or an amino acid sequence that is suitable for use as a dAb) or a Nanobody® (as defined herein, and including but not limited to a V_{HH} sequence); other single variable domains, or any suitable fragment of any one thereof. For a general description of (single) domain antibodies, reference is also made to the prior art cited above, as well as to EP 0 368 684. For the term “dAb's”, reference is for example made to Ward et al. (Nature 1989 Oct. 12; 341 (6242): 544-6), to Holt et al., Trends Biotechnol., 2003, 21(11):484-490; as well as to for example WO 04/068820, WO 06/030220, WO 06/003388 and other published patent applications of Domantis Ltd. It should also be noted that, although less preferred in the context of the present invention because they are not of mammalian origin, single domain antibodies or single variable domains can be derived from certain species of shark (for example, the so-called “IgNAR domains”, see for example WO 05/18629).
   In particular, the amino acid sequence of the invention may be a Nanobody® or a suitable fragment thereof. [Note: Nanobody®, Nanobodies® and Nanoclone® are trademarks of Ablynx N. V.] For a further description of V_{HH}'s and Nanobodies, reference is made to the review article by Muyldermans in Reviews in Molecular Biotechnology 74 (2001), 277-302; as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1 433 793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. Reference is also made to the further prior art mentioned in these applications, and in particular to the list of references mentioned on pages 41-43 of the International application WO 06/040153, which list and references are incorporated herein by reference. As described in these references, Nanobodies (in particular V_{HH} sequences and partially humanized Nanobodies) can in particular be characterized by the presence of one or more “Hallmark residues” in one or more of the framework sequences.
   A further description of the Nanobodies, including humanization and/or camelization of Nanobodies, as well as other modifications, parts or fragments, derivatives or “Nanobody fusions”, multivalent constructs (including some non-limiting examples of linker sequences) and different modifications to increase the half-life of the Nanobodies and their preparations can be found e.g. in WO07/104,529.
w) The term “non-fused” in the context of ‘non-fused dimers’ means every stable linkage (or also more specific conditions herein mentioned as “stable”) existing under normal (e.g. storage and/or physiological) conditions which is not obtained via a direct genetic linkage or via a dedicated dimerization sequence as known in the literature (e.g. Jun-Fos interaction, interaction of CH2-CH3 domains of heavy-chains etc). Such linkage may be due to for example through chemical forces such as Van der Waal's forces, hydrogen bonds, and/or forces between peptides bearing opposite charges of amino acid residues. Furthermore, additional components such as structural changes may play a role. Such structural changes may e.g. be an exchange of framework regions, e.g. exchange of framework region 4 (a phenomenon also called “domain swapping pattern”) beta strands derived from framework regions and may be prevented by stabilizing CDR3-FR4 region in the monomeric structure conformation. In contrast in a genetically linked or -fused construct, the fusion is forcing two entities to be expressed as a fusion protein, and the linkage is of a covalent nature (e.g. using peptide linkers between the two entities, linking the C-terminus of one with the N-terminus of the other protein domain). The term “stable” in the context of “stable dimer” or “stable NFD” (“stable NFDs”) means that 50%, more preferably 60%, more preferably 70%, more preferably 80%, even more preferably 90%, even more preferably 95%, most preferred 99% are in the form of NFDs at the time point of measurement; wherein 100% represents the amount (e.g. molar amount per volume or weight per volume amount) of NFD and its corresponding monomer. Measurement of stability as defined herein, i.e. with regards to its dimeric nature, may be done by using size exclusion chromatography (using standard laboratory conditions such as PBS buffer at room temperature) and if required a pre-concentration step of the sample to be tested. The area under the peak in the size exclusion chromatogram of the identified dimeric and monomeric peak represents the relative amounts of the monomer and dimer, i.e. the NFD. NFD and/or NFDs are used herein interchangeably, thus wherever NFD is used NFDs are meant as well and vice versa.

### Non-Fused-Dimers (NFDs)

Certain conditions or amino acid sequence alterations can convert otherwise stable monomeric single variable domains into stable dimeric and in certain instances multimeric molecules. Key in this process is to provide conditions in which two single variable domains are able to display an increased non-covalent interaction. NFDs are made e.g. in a process called process-induced association (hereinafter also “PIA”). This dimerization is among others a concentration driven event and can e.g. be enhanced by combining high protein concentrations (e.g. higher than 50 mg protein/ml), rapid pH shifts (e.g. pH shift of 2 units within 1 column volume) and/or rapid salt exchanges (e.g. salt exchange with 1 column volume) in the preparation process. The high concentration will enhance the likelihood of interactions of individual monomeric molecules while the pH and salt changes can induce transiently (partial) unfolding and/or promote hydrophobic interactions and/or rearrangement of the protein structure. Because these NFDs may ultimately be used in or as a therapeutic or prognostic agent, the term “NFD” or “NFDs” are meant to mean (or to be interchanged) that the NFD is in solution, e.g. in a physiological preparation, e.g. physiological buffer, comprising NFD or NFDs (unless the condition, e.g. a condition of special sorts, e.g. storage condition for up to 2.5 years, for which a NFD is stable is specifically described). Alternatively. NFDs can also be made under stressful storage conditions e.g. such as relative high temperature (e.g. 37° C.) over weeks such as e.g. 4 weeks. Furthermore. NFDs can be made (even with improved, i.e. faster, kinetics) by introducing destabilizing amino acid residues in the vicinity of the CDR3 and/or the framework region 4 of the singe variable domain susceptible to dimerize (see experimental part, polypeptide F (=mutated polypeptide B) is forming NFDs more quickly than polypeptide B under the same conditions).

Attaining a high concentration of the components that have to dimerize can be obtained with a variety of procedures that include conditions that partially unfold the immunoglobulinic structure of the singe variable domains, e.g. Nanobodies. e.g. via chromatography (e.g. affinity chromatography such as Protein A, ion exchange, immobilized metal affinity chromatography or IMAC and Hydrophobic Interaction Chromatography or HIC), temperature exposure close to the Tm of the single variable domain, and solvents that are unfolding peptides such as 1 to 2 M guanidine. E.g. for chromatography—during the process of elution of the proteins off the column using e.g. a pH shift or salt gradient (as explained later), the NFDs can be formed. Usually the required concentration and/or exact method to form NFDs has to be determined for each polypeptide of the invention and may not be possible for each polypeptide of the invention. It is our experience that there are certain single variable domains either alone (e.g. polypeptides B and F) and/or in a construct (e.g. polypeptides A, C, E, F) that form a NFD. Critical for dimerization may be a relative short CDR3 (e.g. 3 to 8 amino acids, more preferably 4 to 7 amino acids, even more preferably 5 to 6 amino acids, e.g. 6 amino acids)) and destabilizing factors in the vicinity of the CDR3 and/or FR4. Furthermore, high concentration such as e.g. the maximum solubility of the polypeptides comprising single variable domain(s) at the concentration used (e.g. 5 mg polypeptide A per ml protein A resin—see experimental part), or storage at high temperature over weeks (e.g. 37° C. over 4 weeks), low pH (e.g. pH below pH 6), high concentration (higher than 50 mg/ml, e.g. 65 mg/ml) may be required to obtain a reasonable yield of NFD formation.

Next to column chromatography working at e.g. maximum column load, similar required high concentration to obtain NFDs can be achieved by concentration methods such as ultrafiltration and/or diafiltration, e.g. ultrafiltration in low ionic strength buffer.

The process is not linked to a specific number of single variable domains, as the formation of NFDs was observed with monovalent, bivalent and trivalent monomeric building blocks (=polypeptides comprising single variable domain(s)) and even with single variable domain-HSA fusions. In case the polypeptides comprises 2 different single variable domains, NFDs may form via only the identical or different (preferably the identical) single variable domain and usually only via one of the single variable domain(s), e.g. the one identified as susceptible to form NFDs (e.g. polypeptide B) (see also FIG. 2b).

It is an object of the present invention to provide soluble and stable; e.g. stable within a certain concentration range, buffer and/or temperature conditions; dimer-complexes called NFDs that may be used to target molecules and/or thus inhibit or promote cell responses. Herein described are NFDs comprising monomeric building blocks such as single variable domain—also called NFDs-Mo; NFDs comprising dimeric building blocks such as two covalently linked single variable domains—also called NFDs-Di; NFDs comprising trimeric building blocks such as three covalently linked single variable domains—also called NFDs-Tri; NFDs comprising tetrameric building blocks such as four covalently linked single variable domains—also called NFDs-Te; and NFDs comprising more than four multimeric) building blocks such as multimeric covalently linked single variable domains—also called NFDs-Mu (see FIG. 2a+b for schematic overview of such structures). The NFDs may contain identical single variable domains or different single variable domains (FIG. 2b). If the building blocks (polypeptide) consist of different single variable domains, e.g. Nanobodies, it is our experience that preferably only one of the single variable domain in the polypeptide will dimerize. E.g. the dimerizing unit (single variable domain, e.g. Nanobody such as e.g. polypeptide B or F) of a trivalent polypeptide (see FIG. 2b) may be in the middle, at the C-terminus or at the N-terminus of the construct.

It is another object of the invention to provide methods of making and uses to said NFDs.

It is still another object of the present invention to provide information of how to avoid such NFDs.

These above and other objectives are provided for by the present invention which, in a broad sense, is directed to methods, kits, non-fused-dimers that may be used in the treatment of neoplastic, immune or other disorders. To that end, the present invention provides for stable NFDs comprising a single variable domain or single variable domains such as e.g. Nanobody or Nanobodies (e.g. polypeptide B) that may be used to treat patients suffering from a variety of disorders. In this respect, the NFDs of the present invention have been surprisingly found to exhibit biochemical characteristics that make them particularly useful for the treatment of patients, for the diagnostic assessment of a disease in patients and/or disease monitoring assessment in patients in need thereof. More specifically, it was unexpectedly found that certain single variable domains, subgroups thereof (including humanized VHHs or truly camelized human VHs) and formatted versions thereof (and indeed this is also feasible for human VH and derivatives thereof), can be made to form stable dimers (i.e. NFD-Mo, NFD-Di, NFD-Tri. NFD-Te or NFD-Mu) that have beneficial properties with regard e.g. to manufacturability and efficacy. Single variable domains are known to not denature upon for example temperature shift but they reversibly refold upon cooling without aggregation (Ewert et al Biochemistry 2002, 41:3628-36), a hallmark which could contribute to efficient formation of antigen-binding dimers.

NFDs are of particular advantage in many applications. In therapeutic applications, NFDs-Mu, e.g. NDF-Di, binders may be advantageous in situation where oligomerization of the targeted receptors is needed such as e.g. for the death receptors (also referred to as TRAIL receptor). E.g. a NFD-Di due to their close interaction of the respective building blocks are assumed to have a different spatial alignment than “conventional” covalently linked corresponding tetramers and thus may provide positive or negative effect on the antigen-binding (see FIG. 2 for a schematic illustration of certain NFDs). Furthermore, a NFDs, e.g. a NFD-Mo, may bind a multimeric target molecule more effectively than a conventional covalently linked single variable domain dimer. Moreover, heteromeric NFDs may comprise target specific binders and binders to serum proteins, e.g. human serum albumin, with long half life. In addition, “conventional” covalently linked dimers (via e.g. amino acid sequence linkers) may have expression problems (by not having enough tRNA available for certain repetitive codons) and thus it may be advantageous to make the monomers first and than convert the monomers to a NFD in a post-expression process, e.g. by a process described herein. This may give yields that are higher for the NFD compared to the covalently linked dimer. Similarly, it may be expected that e.g. the overall yield of a NFD-Di or NFD-Tri will be higher compared to the relevant covalently linked tetramer or hexamer. The overall higher expression level may be the overriding factor in e.g. cost determination to select the NFD approach. E.g. it is reported that expression yields and secretion efficiency of recombinant proteins are a function of chain size (Skerra & Pluckthun, 1991, Protein Eng. 4, 971).

Moreover, less linker regions could mean less protease susceptible linker regions on the overall protein. It could also be useful to test in vitro and/or in vivo the impact of multimerization of a single variable domain according to the methods described herein. All in all, it is expected that the finding of this invention may provide additional effective solutions in the drug development using formatted single variable domains as the underlying scaffold structure than with the hitherto known approaches, i.e. mainly covalently linked single variable domain formats.

The NFDs of the present invention can be stable in a desirable range of biological relevant conditions such as a wide range of concentration (i.e. usually low nM range), temperature (37 degrees Celsius), time (weeks, e.g. 3 to 4 weeks) and pH (neutral, pH5, pH6 or in stomach pH such as pH 1). In a further embodiment, NFDs of the present invention can be stable (at a rate of e.g. 95% wherein 100% is the amount of monomeric and dimeric form) in vivo, e.g. in a human body, over a prolonged period of time, e.g. 1 to 4 weeks or 1 to 3 months, and up to 6 to 12 months. Furthermore, the NFDs of the present invention can also be stable in a desirable range of storage relevant conditions such as a wide range of concentration (high concentration such as e.g. mg per ml range), temperature (−20 degrees Celsius, 4 degrees Celsius, 20 or 25 degrees Celsius), time (months, years), resistance to organic solvents and detergents (in formulations, processes of obtaining formulations). Furthermore, it has been surprisingly found that denaturation with guanidine HCl (GdnHCl) needs about 1 M more GdnHCl to denature the polypeptide B dimer than the polypeptide B monomer in otherwise same conditions (see experimental part). Additionally, the surprising find that FR4 in the polypeptide B NFD-Mo is swapped (and possibly similarly for other NFDs according to the invention) indicates that indeed this dimers form stable complexes and can further stabilize single variable domain or Nanobody structures. Furthermore, there is evidence that one of the humanisation sites (see experimental part: polypeptide E vs polypeptide B) may have caused a weaker CDR3 interaction with the framework and thus a more extendable CDR3 is available that is more likely to trigger dimerization.

Thus, preferred NFDs of the invention are stable (with regards to the dimeric nature) within the following ranges (and wherein said ranges may further be combined, e.g. 2, 3, 4 or more ranges combined as described below, to form other useful embodiments):
Preferred embodiments of NFDs are stable (with regards to the dimeric nature) under physiological temperature conditions, i.e. temperature around 37 degrees Celsius, over a prolonged time period, e.g. a time up to 1 day, more preferably 1 week, more preferably 2 weeks, even more preferably 3 weeks, most preferred 4 weeks from the time point of delivery of the drug to the patient in need;
Preferred embodiments of NFDs are stable (with regards to the dimeric nature) under various storage temperature conditions, i.e. temperatures such as −20 degrees Celsius, more preferably 4 degrees Celsius, more preferably 20 degrees Celsius, most preferably 25 degrees Celsius, over a prolonged time period, e.g. up to 6 months, more preferably 1 year, most preferred 2 years;
Preferred embodiments of NFDs are stable (with regards to the dimeric nature) under various physiological pH conditions, i.e. pH ranges such as pH 6 to 8, more preferably pH 5 to 8, most preferred pH 1 to 8, over a prolonged time period, e.g. a time up to 1 week, more preferably 2 weeks, even more preferably 3 weeks, most preferred 4 weeks from the time point of delivery of the drug to the patient in need;
Preferred embodiments of NFDs are stable (with regards to the dimeric nature) under various physiological concentration conditions, i.e. concentration of NFDs below 200 ng NFD/ml solvents, e.g. in pH 7 buffer such as phosphate buffered solution and/or e.g. also serum, e.g. human serum; more preferably below 100 ng NFD/ml solvents, even preferably below 50 ng NFD/ml solvents, most preferred 10 ng NFD/ml solvents; in a further preferred embodiment NFDs are stable in above concentrations at 37 degrees Celsius up to 1 day and more, e.g. 1 week, more preferably 2 weeks, more preferably 3 weeks, and most preferred up to 4 weeks;
Preferred embodiments of NFDs are stable (with regards to the dimeric nature) under various physiological concentration conditions, i.e. concentration of NFDs of about 1 mg/ml, more preferably 5 mg/ml, more preferably 10 mg/ml, more preferably 15 mg/ml, more preferably 20 mg/ml, more preferably 30 mg/ml, more preferably 40 mg/ml, more preferably 50 mg/ml, more preferably 60 mg/ml, more preferably 70 mg/ml, and at temperature around 37 degrees Celsius, over a prolonged time period, e.g. a time up to 1 day, more preferably 1 week, more preferably 2 weeks, even more preferably 3 weeks, most preferred 4 weeks from the time point of delivery of the drug to the patient in need;
Preferred embodiments of NFDs are stable (with regards to the dimeric nature) under various storage concentration conditions, i.e. concentration of NFDs above 0.1 mg NFD/ml solvents, e.g. in pH 7 buffer such as phosphate buffered solution: more preferably above 1 mg NFD/ml solvents; more preferably above 5 mg NFD/ml solvents; more preferably above 10 mg NFD/ml solvents, and most preferred above 20 mg NFD/ml solvents; in a further preferred embodiment NFDs are stable in above concentrations at −20 degree Celsius up to 6 months and more, e.g. 1 year, more preferably 2 years, more preferably 3 years, and most preferred up to 4 years: in a further preferred embodiment NFDs are stable in above concentrations at 4 degrees Celsius up to 6 months and more, e.g. 1 year, more preferably 2 years, more preferably 3 years, and most preferred up to 4 years; in a further preferred embodiment NFDs are stable in above concentrations at 25 degrees Celsius up to 6 months and more, e.g. 1 year, more preferably 2 years, more preferably 3 years, and most preferred up to 4 years;
Preferred embodiments of NFDs are stable (with regards to the dimeric nature) in mixtures (e.g. pharmaceutical formulations or process intermediates) with organic solvents, e.g. alcohols such as ethanol, isopropyl alcohol, hexanol and/or others wherein alcohol (preferably ethanol) can be added up to 5%, more preferably 10%, even more preferably 15%, even more preferably 20%, most preferably 30%, for prolonged period of time at a particular temperature, e.g. over long storages, such as at −20 degrees Celsius up to 6 months and more, e.g. 1 year, more preferably 2 years, more preferably 3 years, and most preferred up to 4 years; in a further preferred embodiment NFDs are stable in above mixtures at 4 degrees Celsius up to 6 months and more, e.g. 1 year, more preferably 2 years, more preferably 3 years, and most preferred up to 4 years; in a further preferred embodiment NFDs are stable in above mixtures at 25 degrees Celsius up to 6 months and more, e.g. 1 year, more preferably 2 years, more preferably 3 years, and most preferred up to 4 years, wherein organic solvents such as e.g. alcohol (preferably ethanol) can be added up to 5%, more preferably 10%, even more preferably 15%, even more preferably 20%, most preferably 30%;
Preferred embodiments of NFDs are stable (with regards to the dimeric nature) in mixtures (e.g. pharmaceutical formulations or process intermediates) with detergents, e.g. non-ionic detergents such as e.g. Triton-X, up to 0.01%, more preferably 0.1%, most preferably 1%, for prolonged period of time at a particular temperature, e.g. over long storages, such as at −20 degrees Celsius up to 6 months and more, e.g. 1 year, more preferably 2 years, more preferably 3 years, and most preferred up to 4 years; in a further preferred embodiment NFDs are stable in above mixtures at 4 degrees Celsius up to 6 months and more, e.g. 1 year, more preferably 2 years, more preferably 3 years, and most preferred up to 4 years; in a further preferred embodiment NFDs are stable in above mixtures at 25 degrees Celsius up to 6 months and more, e.g. 1 year, more preferably 2 years, more preferably 3 years, and most preferred up to 4 years.

Another embodiment of the current invention is that the NFDs retain the binding affinity of at least one of the two components compared to the monomers, e.g. said affinity or of the NFDs may be not less than 10%, more preferably not less than 50%, more preferably not less than 60%, more preferably not less than 70%, more preferably not less than 80%, or even more preferably not less than 90% of the binding affinity of the original monomeric polypeptide; or it has multiple functional binding components, with apparent affinity improved compared to the monomer, e.g. it may have a 2 fold, 3, 4, 5, 6, 7, 8, 9 or 10 fold, more preferably 50 fold, more preferably 100 fold more preferably 1000 fold improved affinity compared to the original monomeric polypeptide.

Another embodiment of the current invention is that the NFDs partially or fully loose the binding affinity of at least one of the two components compared to the monomers, e.g. said affinity or of the NFDs may be not less than 90%, more preferably not less than 80%, more preferably not less than 70%, more preferably not less than 60%, more preferably not less than 50%, even more preferably not less than 30%, even more preferably not less than 20%, even more preferably not less than 10%, or even more preferably not less than 1% of the binding affinity of the original monomeric polypeptide or most preferred the binding affinity may not be detectable at all; or it has multiple functional binding components, with apparent affinity compared to the monomer that is decreased, e.g. it may have a 2 fold, 3, 4, 5, 6, 7, 8, 9 or 10 fold, more preferably 50 fold, more preferably 100 fold more preferably 1000 fold decreased affinity compared to the original monomeric polypeptide.

Furthermore, an embodiment of the current invention is a preparation comprising NFDs and their monomeric building blocks, e.g. preparations comprising more than 30% NFDs (e.g. the 2 identical monomeric building blocks that form said NFD), e.g. more preferably preparations comprising more than 35% NFDs, even more preferably preparations comprising more than 40% NFDs, even more preferably preparations comprising more than 50% NFDs, even more preferably preparations comprising more than 60% NFDs, even more preferably preparations comprising more than 70% NFDs, even more preferably preparations comprising more than 80% NFDs, even more preferably preparations comprising more than 90% NFDs, even more preferably preparations comprising more than 95% NFDs, and/or most preferred preparations comprising more than 99% NFDs (wherein 100% represents the total amount of NFDs and its corresponding monomeric unit). In a preferred embodiment, said ratios in a preparation can be determined as e.g. described herein for NFDs.

Moreover, another embodiment of the current invention is a pharmaceutical composition comprising NFDs, more preferably comprising more than 30% NFDs (e.g. the 2 identical monomeric building blocks form said NFD), e.g. more preferably a pharmaceutical composition comprising more than 35% NFDs, even more preferably a pharmaceutical composition comprising more than 40% NFDs, even more preferably a pharmaceutical composition comprising more than 50% NFDs, even more preferably a pharmaceutical composition comprising more than 60% NFDs, even more preferably a pharmaceutical composition comprising more than 70% NFDs, even more preferably a pharmaceutical composition comprising more than 80% NFDs, even more preferably a pharmaceutical composition comprising more than 90% NFDs, even more preferably a pharmaceutical composition comprising more than 95% NFDs, and/or most preferred a pharmaceutical composition comprising more than 99% NFDs (wherein 100% represents the total amount of NFDs and its corresponding monomeric unit).

Another embodiment of the present invention is a mixture comprising polypeptides in monomeric and dimeric form, i.e. the NFDs, wherein said preparation is stable for 1 months at 4 degrees Celsius in a neutral pH buffer in a 1 mM, more preferably 0.1 mM, more preferably 0.01 mM, more preferably 0.001 mM, or most preferably 100 nM overall concentration of monomeric and dimeric form), and wherein said preparation comprises more than 25%, more preferably 30%, more preferably 40%, more preferably 50%, more preferably 60%, more preferably 70%, more preferably 80% or more preferably 90% dimer, i.e. NFD.

While the methodology described here, is or may in principle applicable to dimerize or multimerize either Fab fragments, Fv fragments, scFv fragments or single variable domains, it is the latter for which their use is most advantageous. In this case dimeric fragments, i.e. the NFDs, can be constructed that are stable, well defined and extend the applicability of said single variable domains beyond the current horizon. In a preferred embodiment, the NFDs are obtainable from naturally derived VHH, e.g. from Llamas or camels, according to the methods described herein or from humanized versions thereof, in particular humanized versions wherein certain so called hallmark residues, e.g. the ones forming the former light chain interface residues, also e.g. described in WO 2006/122825, or in FIG. 1 herein, are not changed and stay as derived from the naturally obtained single variable domains. In a further preferred embodiment, the NFDs are obtainable from polypeptides comprising at least a single domain antibody (or Nanobody) with similar CDR3 and FR4 amino acid residues (SEQ ID NO: 9) as polypeptide B, e.g. NFDs obtainable from polypeptides comprising at least a Nanobody having a CDR3 and FR4 region that has a 80%, more preferably 90%, even more preferably 95%, 96%, 97%, 98%. 99% sequence identity to SEQ ID NO: 9.

Previously, increasing the number of binding sites based on single variable domains meant the preparation of covalently linked domains at the genetic level or via other interaction domains (e.g. via fusion to Fc, Jun-Fos, CH2/CH3 constant domain of heavy chain interaction, VL-VH antibody domain interactions etc), whereas now it is possible to alternatively form such entities later, at the protein level. These non-fused dimers combine three main features: (a) possibility to combine one or more single variable domains of one or more specificities (e.g. against target molecule and against serum protein with long half life) into NFDs by biochemical methods (vs genetic methods), (b) controlled dimeric interaction that retains or abolishes antigen binding (vs “uncontrolled” aggregation), and (c) stability sufficient e.g. for long term storage (for practical and economic reasons) and application in vivo, i.e. for application over prolonged time at e.g. 37 degrees Celsius (important requirement for the commercial use of these NFDs).

Thus, it is a further object of the invention to create new individual and stable NFDs with bi- or even multifunctional binding sites. It has been found that antibody fragment fusion proteins containing single variable domains could be produced by biochemical methods which e.g. show the specified and improved properties as described herein. For example, a particular embodiment of the present invention is a NFD or NFDs comprising a first polypeptide comprising single variable domain(s), e.g. a Nanobody or Nanobodies, against a target molecule and a second polypeptide comprising single variable domain(s), e.g. a Nanobody or Nanobodies, against a serum protein, e.g. human serum albumin (see e.g. polypeptide C and E (each binding a receptor target and human serum albumin) in the experimental part, see also FIG. 2a+b). Other examples of using bispecificity can be found in Kufer et al, Trends in Immunology 22: 238 (2004). In the case in which two different antigen-binding single variable domains are used, the procedure to produce NFDs may be tweaked to promote the formation of heterodimers versus homodimers, or alternatively be followed by a procedure to separate these forms.

Moreover, it is an object of the invention, therefore, to provide (or select) in a first step a monomeric polypeptide essentially consisting of a single variable domain, wherein said polypeptide is capable to dimerize with itself by process-induced association (PIA) or other alternative methods described herein.

More specifically, we describe in this invention NFDs obtainable by e.g. a method that comprises the step of screening for preparations comprising antibody fragments or polypeptides comprising single variable domain(s) that form dimers by the processes as described herein. Hence said screening method comprising identifying said polypeptides may be a first step in the generation of NFDs. Multiple ‘PIA’ methods described herein can be used to force dimer formation in a starting preparation comprising its monomeric building block. At this point an indication that dimers may be formed under suitable conditions, e.g. the process induced association (PIA) as described herein. An indication is sufficient at this time and may simply mean that a small amount of e.g. the protein A purified fraction in the size exclusion chromatography is eluting as a presumable dimer in the standard purification protocol. Once the dimerization is suggested and later confirmed (e.g. by analytical SEC, dynamic light scattering and/or analytical ultracentrifugation) further improvement in order to favour dimerization (e.g. by higher column load, conditions favouring partial unfolding, conditions favouring hydrophobic interactions, high temperature such as e.g. 37° C. exposure of some time, e.g. weeks such as e.g. 4 weeks, introduction of CDR3 destabilizing amino acid residues etc) or in order to minimize dimerization (opposite strategy) can be initiated (in order to e.g. increase the yield).

The invention relates, furthermore, to a process of selection of a monomeric polypeptide that comprises at least one single variable domain, preferably at least one Nanobody, capable of forming a NFD according to the invention and as defined herein, characterized in that the NFD is stable and preferably has a similar or better apparent affinity to the target molecule than the monomeric polypeptide showing that the binding site is active or at least is partially active. Said affinity may be not less than 10%, more preferably 50%, more preferably not less than 60%, more preferably not less than 70%, more preferably not less than 80%, or even more preferably not less than 90% of the binding affinity of the original monomeric polypeptide, e.g. may have a 2 fold, 3, 4, 5, 6, 7, 8, 9 or 10 fold, more preferably 50 fold, more preferably 100 fold more preferably 1000 fold improved apparent affinity compared to original monomeric polypeptide. Said affinity may be expressed by features known in the art, e.g. by dissociation constants, i.e. Kd, affinity constants, i.e. Ka, koff and/or kon values—these and others can reasonably describe the binding strength of a NFD to its target molecule.

Moreover, the invention relates, furthermore, to a process of selection of a monomeric polypeptide that comprises at least one single variable domain, preferably at least one Nanobody, capable of forming a NFD according to the invention and as defined herein, characterized in that the NFD is stable and preferably has no apparent affinity to the target molecule, e.g. human serum albumin.

Said selection may comprise the step of concentrating the preparation comprising the monomeric starting material, i.e. the polypeptide comprising or essentially consisting of at least one single variable domain, to high concentration, e.g. concentration above 5 mg/ml resin, by methods known by the skilled person in the art, e.g. by loading said polypeptide to a column, e.g. protein A column, to the near overload of the column capacity (e.g. up to 2 to 5 mg polypeptide per ml resin protein A) and then optionally eluting said polypeptide with a “steep” pH shift (“steep” meaning e.g. a particular pH shift or change (e.g. a decrease or increase of 10, more preferably 100 or more preferably 1000 fold of the H+ concentration) in one step (i.e. immediate buffer change) or within one, two or three (more preferably one or immediate buffer change) column volume(s)). Furthermore, the “steep” pH shift may be combined with a selected pH change, i.e. the pH can start above or below the pI of the polypeptide and then change into a pH below or above the pI of said polypeptide. Alternatively, concentration of said polypeptides leading to NFD formation is obtainable by other means such as e.g. immobilized metal ion affinity chromatography (IMAC), or ultra-filtration. Preferably conditions are used wherein the polypeptides of the invention are likely to unfold (extremes in pH and high temperature) and/or combinations of conditions favouring hydrophobic interaction such as e.g. pH changes around the pI of the polypeptide and low salt concentration. Furthermore, the conditions used to drive these dimers apart may be also useful to explore when determining further methods for producing these dimers, i.e. combining these procedures (e.g. 15 minutes of exposure to a temperature of about 70 degrees Celsius for Polypeptide A with a high polypeptide concentration and subsequent cooling).

Examples of methods to obtain NFDs are further described in a non limiting manner in the experimental part of this invention.

Another object of the invention is the process to obtain a NFD characterized in that the genes coding for the complete monomeric polypeptide comprising at least one single variable domain (e.g. one, two, three or four single variable domain(s)) or functional parts of the single variable domain(s) (e.g. as obtained by the screening method described herein) are cloned at least into one expression plasmid, a host cell is transformed with said expression plasmid(s) and cultivated in a nutrient solution, and said monomeric polypeptide is expressed in the cell or into the medium, and in the case that only parts of the fusion proteins were cloned, protein engineering steps are additionally performed according to standard techniques.

Furthermore, another object of the invention is the process of associating two monomeric identical polypeptides comprising at least one single variable domain (e.g. one, two, three or four single variable domain(s)) or functional parts of the single variable domain(s) to form a NFD, wherein said process comprises the step of creating an environment where hydrophobic interactions and/or partial refolding of said polypeptides are favoured e.g. by up-concentrating a preparation comprising the monomeric polypeptides, salting-out, adding detergents or organic solvents, neutralizing the overall charge of said polypeptide (i.e. pH of polypeptide solution around the pI of said polypeptide or polypeptides) and/or high temperature close to the melting temperature of the polypeptide or the single variable domain susceptible to dimerization, e.g. at temperature around 37° C. or higher e.g. 40° C., 45° C. or 50° C. or higher over a prolonged time, e.g. weeks such as e.g. 1, 2 3, 4 or more weeks, preferably 4 weeks during dimerization process thus allowing close interaction between the polypeptides. Interestingly and surprisingly said conditions do not have to be upheld in order to stabilize the NFDs once the dimer is formed, i.e. the NFDs in solution are surprisingly stable in a wide range of biological relevant conditions such as mentioned herein.

The NFDs according to the invention may show a high avidity against corresponding antigens and a satisfying stability. These novel NFD structures can e.g. easily be prepared during the purification process from the mixture of polypeptides and other proteins and/or peptides obtained by the genetically modified prokaryotic or eukaryotic host cell such as e.g. E. coli and Pichia pastoris.

Furthermore, the monomeric building blocks capable of forming NFDs may be pre-selected before doing a process for selection or screening as above and further herein described by taking into consideration primary amino acid sequences and crystal structure information if available. Moreover, in order to understand the potential interactions in these non-fused protein domains, it may be advisable to analyze different X-ray or NMR structures of non-fused single variable domains, i.e. NFDs. This then exemplifies how possibly in solution interactions in NFDs can occur but this is by no means then a complete explanation for the likely area of interaction between the NFD components.

Furthermore, further stabilization of the dimer may be beneficial and may be done by suitable linker linking the ends of the polypeptides and/or cysteines at the interaction sites. E.g. a covalent attachment of the two domains may be possible by introducing 2 cysteines in each of the two building blocks at spatially opposite positions to force formation of a disulphide bridge at the new site of interaction, or at N- or C-terminal region of the NFD as has e.g. been done with diabodies (Holliger & Hudson, Nat Biotech 2004, 23 (9): 1126. Furthermore, it may be advantageous to introduce a flexible peptide between the ends of the two monomeric building blocks. As an example, the upper hinge region of mouse IgG3 may be used. However, a variety of hinges or other linkers may be used. It is not required for dimerization per se, but provides a locking of the two building blocks. The naturally occurring hinges of antibodies are reasonable embodiments of hinges. In such case, the polypeptides of the invention need to be present first under reducing conditions, to allow the NFDs to form during purification after which oxidation can lead to the cysteine pairings, locking the NFDs into a fixed state. In the case of NFDs, the hinges or linkers may be shorter than in conventional covalently linked single variable domain containing polypeptides. This is not to disturb the expected close interaction of the monomeric building blocks, and flexibility of the dimer is not necessary. The choice of the hinge is governed by the desired residue sequence length (Argos, 1990, J. Mol. Biol. 211, 943-958), compatibility with folding and stability of the dimers (Richardson & Richardson, 1988, Science 240, 1648-1652), secretion and resistance against proteases, and can be determined or optimized experimentally if needed.

Furthermore, further stabilization of the monomers may be beneficial (i.e. avoidance of the dimerization or in certain instances possible multimerizations) and may be done by choosing suitable linkers linking the ends of the polypeptides and/or cysteines at or close to the CDR3 and/or FR4 region that prevent the single variable domain from dimerization. E.g. a covalent stabilization of the CDR3 and/or FR4 may be possible by introducing 2 cysteines close to or/and within the CDR3 and/or FR4 region at spatially opposite positions to force formation of a disulphide bridge as has e.g. been done with cystatin that was stabilized against three-dimensional domain swapping by engineered disulfide bonds (Wahlbom et al., J. of Biological Chemistry Vol. 282, No. 25, pp. 18318-18326, Jun. 22, 2007). Furthermore, it may be advantageous to introduce a flexible peptide that is then engineered to have one cysteine that than forms a disulfide bond to e.g. a cysteine before the CDR3 region. In such case, the polypeptides of the invention need to be present first under reducing conditions, to allow the monomers to form after which oxidation can lead to the cysteine pairings, locking the monomers into a fixed, stabilized state.

Furthermore, further stabilization of the monomers may be beneficial (i.e. avoidance of the dimerization or in certain instances possible multimerizations) and may be done by replacing a destabilizing amino acid residue or residues (e.g. identified, by screening of mutants, e.g. by affinity maturation methods—see e.g. WO2009/004065) by a stabilizing amino acid residue or residues in the vicinity of CDR3 and/or FR4.

In an other aspect of the invention, further stabilization of the monomers can be achieved (i.e. avoidance of the dimerization or in certain instances possible multimerizations) by suitable formulation. In particular, the present invention provides a method for suppressing the dimerization and multimerization of (human serum) albumin-binding Nanobodies (e.g. polypeptide B) and other polypeptides comprising Nanobodies by providing mannitol or other polyols to a liquid formulation. Mannitol is generally used for maintaining the stability and isotonicity of liquid protein formulations. It is also a common hulking agent for lyophilization of the formulation. Surprisingly, the present invention discovered that mannitol can specifically inhibit the formation of dimers observed during storage (at elevated temperature) of several albumin-binding Nanobodies. As a result, mannitol-containing formulations increase protein stability and sustain biological activity, thereby prolonging the shelf-life of the drug product. The stabilizing effect of mannitol is supported by data that demonstrate higher Tm (melting temperature) values in protein formulations with increasing mannitol concentrations.

This invention will also cover the use of other polyols, non-reducing sugars, NaCl or amino acids.

The dimers formed by e.g. the serum albumin-binding Nanobody “polypeptide B” of the invention (SEQ ID NO: 2) was shown to be completely inactive for binding to HSA (Biacore analysis), suggesting that the albumin binding site in the dimer interface is blocked by dimer formation. The addition of mannitol to the liquid formulation as proposed by this invention will therefore not only suppress the dimerization process but, importantly, will also preserve the HSA-binding activity of Nanobody and slow down the inactivation. In general, the Mannitol containing formulations according to the inventions prolong the shelf-life of the formulated protein/drug product. The invention is believed to be applicable to any albumin-binding Nanobody and may be applicable to all. Nanobodies that have a tendency to form dimers in general. Thus, the Mannitol formulations of the invention are indicated for the formulation of any Nanobody, as process intermediate, drug substance or drug product. This invention may be used in a wide variety of liquid formulations which may consist of any buffering agent, a biologically effective amount of protein, a concentration of mannitol that is no greater than approximately 0.6M and other excipients including polyols, non-reducing sugars, NaCl or amino acids. The liquid formulations may be stored directly for later use or may be prepared in a dried form, e.g. by lyophilization. Mannitol may be used in any formulation to inhibit the formation of high molecular weight species such as the observed dimers during storage, freezing, thawing and reconstitution after lyophilization.

A particular advantage of the NFDs described in this invention is the ability to assemble functionally or partly functionally during e.g. the manufacturing process (e.g. purification step etc) in a controllable manner. A dimerization principle is used which allows the formation of homodimers. Examples described herein include NFDs-Mo, NFDs-Di, and NFDs-Tri. In these cases, the monomeric building blocks are expressed in a bacterial system and then bound in high concentration to a separation chromatographic device, e.g. Protein A or IMAC, and eluted swiftly to retain the desired dimeric complexes, i.e. the NFDs, in substantial yield. Under these conditions, the homodimeric proteins form by themselves and can directly be isolated in the dimeric form by said separation step and/or further isolated by size exclusion chromatography.

### SHORT DESCRIPTION OF THE FIGURES

FIG. 1: Hallmark Residues in single variable domains.

FIG. 2a+b: Illustration of various non-fused dimers (i.e. NFDs) and comparison with the conventional genetically fused molecules. Single Variable Domains in each construct or NFD may be different (2a+b) or identical (2a). The dashed line is a schematic interaction between the 2 VH domains that confer the NFD its stability (indicated here are surface interactions but these can also be other interaction as described in the invention herein).

FIG. 3: Protein A affinity purification of polypeptide A (SEQ ID NO: 1) under conditions resulting in significant amounts of NFDs.

The protein was loaded on a small column (400 μl resin MabSelectXtra, GE Healthcare) and eluted via injection of glycine [100 mM, pH=2.5]. The pH of the eluted Nanobody® solution was immediately neutralized using 1M Tris pH 8.8.

FIG. 4: Size exclusion chromatography of Protein A affinity purified of polypeptide A. Separation of concentrated polypeptide A (fraction 6, see FIG. 3) on an analytical Superdex 75 column (GE Healthcare). The Nanobody fraction is resolved into two specific fractions corresponding to the molecular weight of monomeric and dimeric polypeptide A (position of molecular weight markers is indicated).

Analysis via SDS-PAGE (right panel) did not reveal any difference between the two, indicating that under native conditions they behave as monomer and dimer. The latter is converted into a monomer conformation upon denaturation (SDS detergent and heat treatment).

FIG. 5: Protein A affinity purification of polypeptide A at low column load.

A limited amount of protein [approx. 2.5 mg/ml resin] was loaded on a small column (400 μl resin MabSelectXtra, GE Healthcare) and eluted via injection of glycine [100 mM, pH=2.5]. The pH of the eluted Nanobody® solution was immediately neutralized using 1M Tris pH 8.8.

FIG. 6: Size exclusion chromatography of Protein A affinity purified of polypeptide A. Separation of concentrated polypeptide A (fraction 7, see FIG. 5) on an analytical Superdex 75 column (GE Healthcare). The Nanobody fraction is resolved into a specific fractions corresponding to the molecular weight of monomeric polypeptide.

FIG. 7: Protein A elution of Polypeptide A. The pretreated periplasmic extract was loaded on a Protein A MabSelectXtra column, followed by a PBS wash until stable baseline. Elution was carried out via a pH shift using 100 mM glycin pH=2.5 (dotted line).

FIG. 8: Size Exclusion Chromatography of Polypeptide A monomer and dimer. The pre-peak (fraction 2) contains the dimeric Polypeptide A which was used in the stability studies.

FIG. 9: Size exclusion chromatography of heat treated samples of dimeric Polypeptide A. Polypeptide A NFD (at 0.68 mg/ml) was used in several experiments: 20 μl dimer fractions were diluted with 90 μl D-PBS and incubated at different temperatures and 100 μl was analysed on a Superdex 75™ 10/300GL column equilibrated in D-PBS.

FIG. 10: Size exclusion chromatography of pH treated samples of Polypeptide A NFD. Polypeptide A NFD (at 0.68 mg/ml) was used in several experiments: 20 μl dimer samples were diluted with [100 mM piperazin pH=10.2] or 90 μl [100 mM Glycin, pH=2.5] and incubated overnight (ON) at 4° C. The control was incubated in D-PBS. Samples were analysed via SEC the next day. The incubation at elevated pH had no effect on the dissociation whereas low pH (glycin pH=2.5) resulted in approx 15% monomer. A more drastic incubation in 1% TFA during 15 min at room temperature resulted in almost 100% monomer.

FIG. 11: Size exclusion chromatography of combined heat/organic solvent treated samples of Polypeptide A NFD. Polypeptide A NFD (at 0.68 mg/ml) was used in several experiments: 20 μl dimer fractions were diluted with [10% Isopropanol] or 90 μl [30% Isopropanol] and incubated overnight (ON) at 4° C. or 15 minutes at 20° C. Combined treatments (heat and Isopropanol) were carried out during 15 minutes. The control was incubated in D-PBS. Samples were analysed via SEC. The incubation at elevated temperature with organic solvent resulted in accelerated dissociation into monomer.

FIG. 12: Size exclusion chromatography of ligand-NFD complex formation: 20 μl samples of Ligand A (SEQ ID NO: 6) was diluted in 90 μl [HBS-EP (Biacore)+0.5M NaCl] and incubated for several hours at RT (ligand mix). Then NFD or Polypeptide A was added and after a short incubation (typically 30 min) the material was resolved via SEC. Polypeptide A [3.91 mg/ml]: 17 μl [ 1/10 diluted in HBS-EP] was added to the ligand mix and 100 μl was injected.

FIG. 13: The molecular weight (MW) of polypeptide A. Ligand A, Polypeptide A+Ligand A, NFD-Di of Polypeptide A, and NFD-Di of Polypeptide A+Ligand A was calculated (see Table 2 for read out from this figure) based on curve fitting of Molecular weight standards (Biorad #151-1901) run on the same column under same conditions.

FIG. 14: monomer A as present in the dimer (top) and an isolated monomer of polypeptide B (bottom)

FIG. 15: Polypeptide B-dimer (an example of a NFD-Mo). Framework 4 of monomer A is replaced by framework 4 of monomer B and vice versa.

FIG. 16: Electron-density of monomer B in black. Monomer A is shown in grey ribbon.

FIG. 17: Polypeptide B (top) and polypeptide F with Pro at position 45 (bottom).

FIG. 18: Size exclusion chromatography of material eluted from Protein A affinity column on Superdex 75 XK 26/60 column.

FIG. 19: Fluorescence emission Sypro orange in the presence of polypeptide B and polypeptide B-dimer (Alb11=polypeptide B).

FIG. 20: Unfolding of Polypeptide B (=Alb11) monomer and Polypeptide B-dimer (=Alb11-dimer) in function of guanidine concentration. Unfolding was monitored by intrinsic fluorescence measurements and thereby using CSM as unfolding parameter.

FIG. 21: Purity was analysed on a Coomassie stained gel (Panel A: Polypeptide G; Panel B: Polypeptide H)

FIG. 22: Binding of polypeptide F, G, and H on HSA

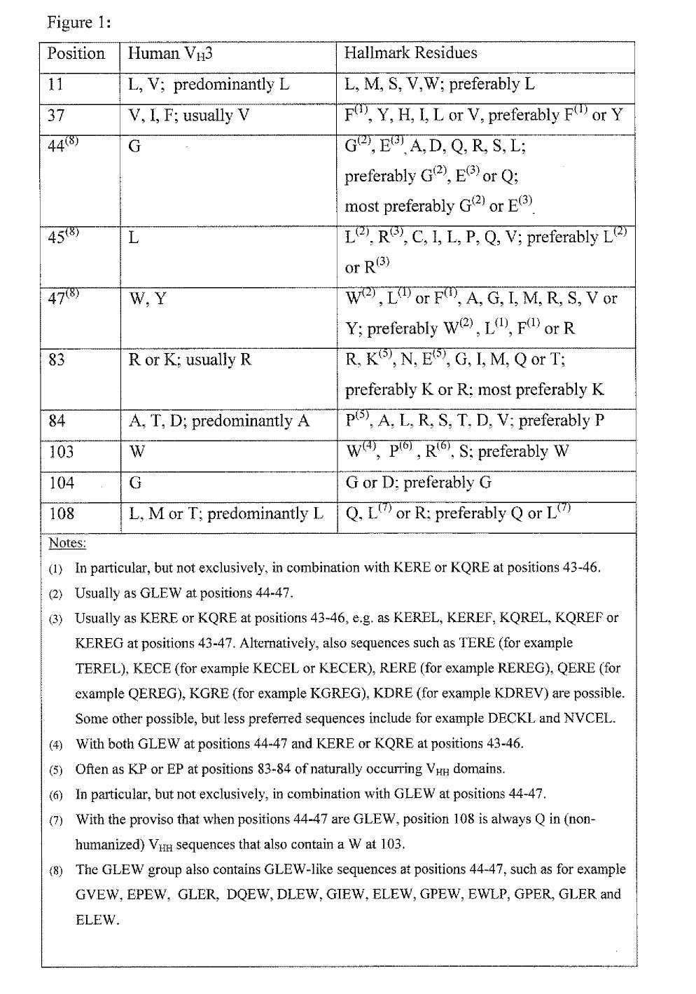

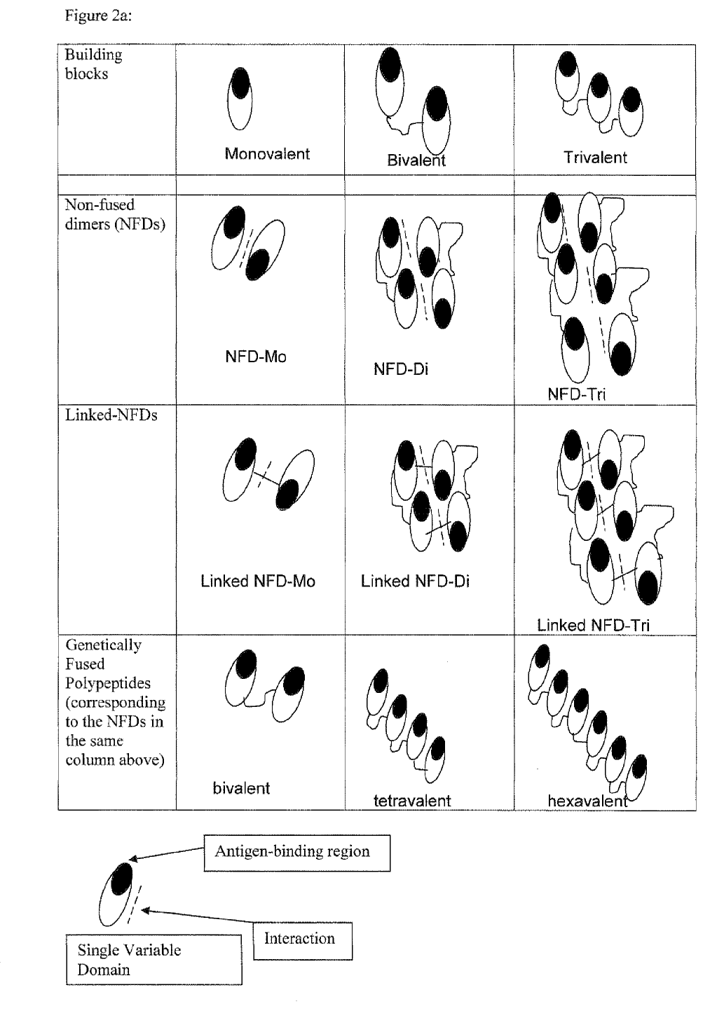

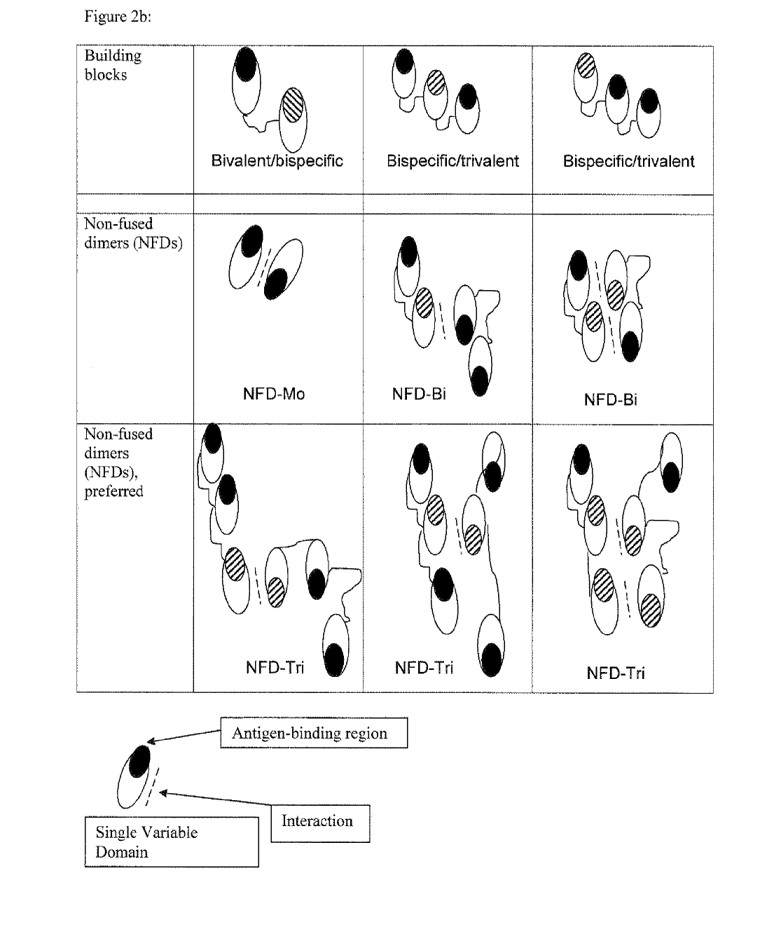

### EXPERIMENTAL PART

### Example 1

### Generation of NFDs

Fermentation of Polypeptide A (SEQ ID NO: 1) Producing E. coli Clone.

Fermentation of Polypeptide A (SEQ ID NO: 1) clone1 (identified as disclosed in WO 2006/122825) was carried out at 10 liter scale in Terrific Broth (Biostat Bplus, Sartorius) with 100 μg/ml carbenicillin. A two percent inoculum of the preculture (grown overnight in TB, 2% glucose. 100 μg/ml carbenicillin) was used to start the production culture (22° C./lvvm). Induction (using 1 mm IPTG) was started at an OD₆₀₀ of 8.0. After a short induction at 22° C. the cell paste was collected via centrifugation (Sigma 8K, rotor 12510; 7000 rpm for 30 min) and frozen at −20° C.

### Purification of Polypeptide A.

Purified Polypeptide A (monomer and dimer) was generated via a process consisting of 6 steps:

1. Extraction from Cell Pellet

The frozen cell pellet was thawed, the cells were resuspended in cold PBS using an Ultra Turrax (Ika Works; S25N-25G probe, 11.000 rpm.) and agitated for 1 h at 4° C. This first periplasmic extract was collected via centrifugation; a second extraction was carried out in a similar way on the obtained cell pellet. Both extractions did account for more than 90% of the periplasmic Polypeptide A content (the 2^{nd} extraction did yield about 25%).

### 2. Removal of Major Contaminants Via Acidification

The periplasmic extract was acidified to pH=3.5 using 1M citric acid (VWR (Merck) #1.00244.0500) 10 mM molar final pH=3.5 and further pH adjusted with 1M HCl. The solution was agitated overnight at 4° C. The precipitated proteins and debris was pelleted down via centrifugation.

### 3. Micro-Filtration and Concentration of the Extract

The supernatant was made particle free using a Sartocon Slice Crossflow system (17521-101, Sartorius) equipped with Hydrosart 0.20 μm membrane (305186070 10-SG, Sartorius) and further prepared for Cation Exchange Chromatography (CEX) via Ultra filtration. The volume that needed to be applied to CEX was brought down to approx 2 liter via ultra filtration using a Sartocon Slice Crossflow system equipped with Hydrosart 10,000MWCO membranes (305144390 1E-SG, Sartorius). At that point the conductivity (<5 mS/cm) and pH (=3.5) were checked.

### 4. Capture and Purification Via CEX

The cleared and acidified supernatant was applied to a Source 30S column (17-1273-01, GE Healthcare) equilibrated in buffer A (10 mM Citric acid pH=3.5) and the bound proteins were eluted with a 10CV linear gradient to 100% (1M NaCl in PBS). The Polypeptide A fraction was collected and stored at 4° C.

### 5. Affinity Purification on Protein A Column

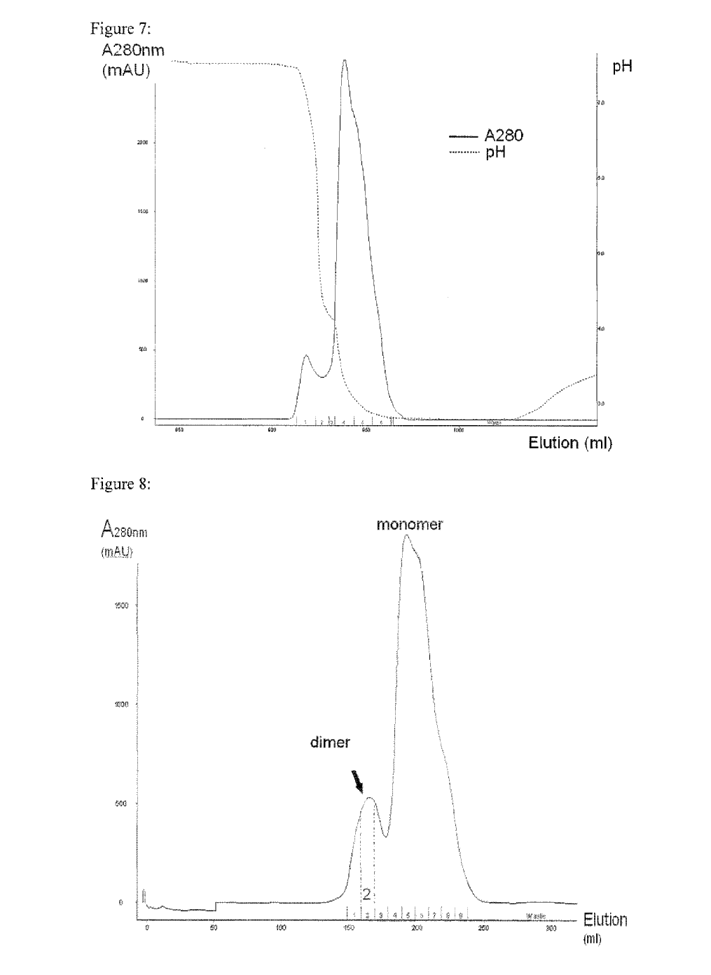

Polypeptide A (amount=well below column capacity) was further purified via Protein A affinity chromatography (MabSelect Xtra™, 17-5269-07, GE Healthcare). A one step elution was carried out using 100 mM Glycine pH 2.5. The collected sample was immediately neutralized using 1M Iris pH7.5 (see FIG. 7).

6. Size Exclusion Chromatography (Optional e.g. in Order to Isolate NFDs and/or Determine Amount of NFDs)

The purified Nanobody® fraction was further separated and transferred to D-PBS (Gibco#14190-169) via SEC using a Hiload™ XK26/60 Superdex 75 column (17-1070-01, GE Healthcare) equilibrated in. D-PBS. Fraction 2 contained the dimeric Polypeptide A (see FIG. 8).

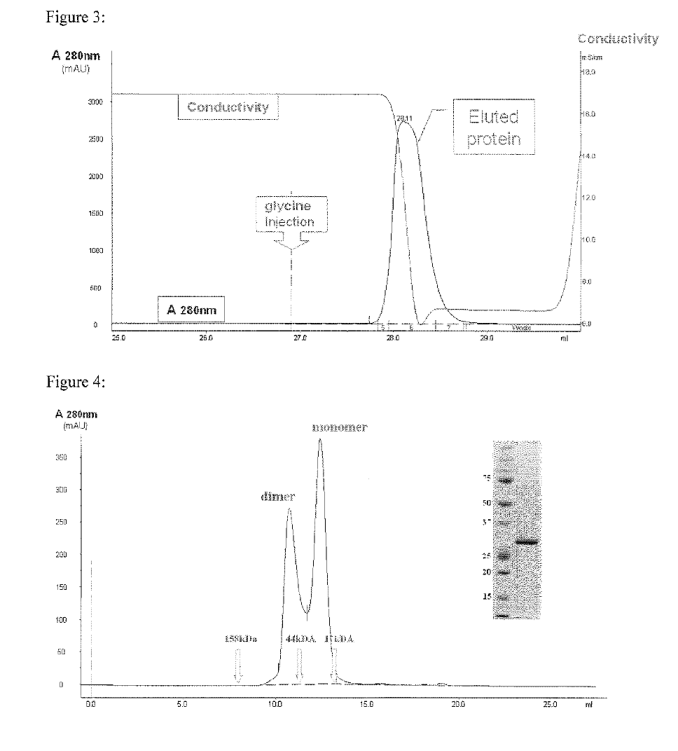

In a further experiment, Polypeptide A (SEQ ID NO: 1) was accumulated on a Protein A column, its concentration well above 5 mg polypeptide A/ml resin, and eluted via a steep pH shift (one step buffer change to 100 mM Glycine pH 2.5). During elution of the polypeptide A from the column it was ‘stacked’ into an elution front, consisting of ‘locally’ very high concentrations (actual value after elution >5 mg/ml), and combination with the pH shift led to the isolation of about 50% stable dimer (see FIG. 3).

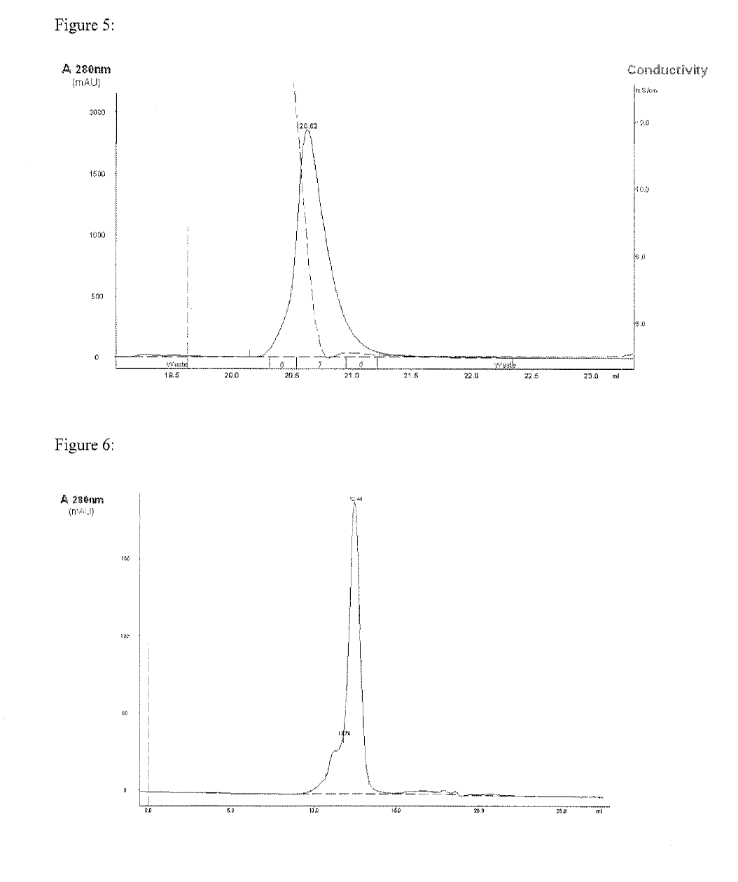

The shift from monomer to dimer is demonstrated via size exclusion chromatography (SEC), allowing determination of the percentage of dimerization (see FIG. 4). When loading less polypeptide A on Protein A (i.e. 2 mg/ml resin under otherwise same conditions as above, i.e. one step elution with 100 mM Glycine pH 2.5), almost no dimers (<5%) were detected during SEC (see FIG. 5 and FIG. 6). Similarly, NFDs of a polypeptide comprising one singe variable domain (NFD-Mo), a polypeptide comprising three single variable domains (NFD-Tri), and a polypeptide comprising a HSA (human serum albumin) and a single variable domain fusion were obtained (see Table 1).

TABLE 1
Examples of obtained NFDs

| Code for | SEQ ID NO of | | Isolated | Monomeric |
|---|---|---|---|---|
| Monomeric | monomeric | | stable NFD | polypeptide |
| polypeptide | building block | Obtained by | type | comprising |
| Polypeptide | 1 | Protein | NFD-Di | Two identical singe |
| A | | A + SEC | | variable domains |
| Polypeptide | 2 | IMAC + AEX + | NFD-Mo | One single variable |
| B, also | | SEC; | | domain binding to |
| referred to as | | Protein | | human serum |
| Alb11 | | A + SEC | | albumin |
| Polypeptide | 3 | Protein | NFD-Tri | Three single |
| C | | A + SEC | | variable domains of |
| | | | | which one binds to |
| | | | | human serum |
| | | | | albumin and the 2 |
| | | | | other single |
| | | | | variable domains to |
| | | | | a receptor target |
| Polypeptide | 4 | Protein | NFD-Mo | Singe variable |
| D | | A + SEC | | domain and HSA |
| Polypeptide | 5 | Protein | NFD-Di | Two single variable |
| E | | A + SEC | | domains of which |
| | | | | one binds to human |
| | | | | serum albumin and |
| | | | | the other single |
| | | | | variable domain to |
| | | | | a receptor target |
| Polypeptide | 6 | Protein | NFD-Mo | One single variable |
| F, also | | A + SEC | | domain binding to |
| referred to as | | | | human serum |
| Alb11 | | | | albumin |

### Example 2

### Stability of NFDs

During purification of Polypeptide A stable non fused dimers (NFDs) were generated (see above). In order to get more insight into the stability and nature of this non-covalent interaction, stable Polypeptide A NFDs were subjected to distinctive conditions aiming to dissociate the dimer into monomer. The stability of the complex was evaluated via 3 criteria: heat-stability, pH-stability, organic solvent resistance and combinations thereof.

### Experimental Set Up

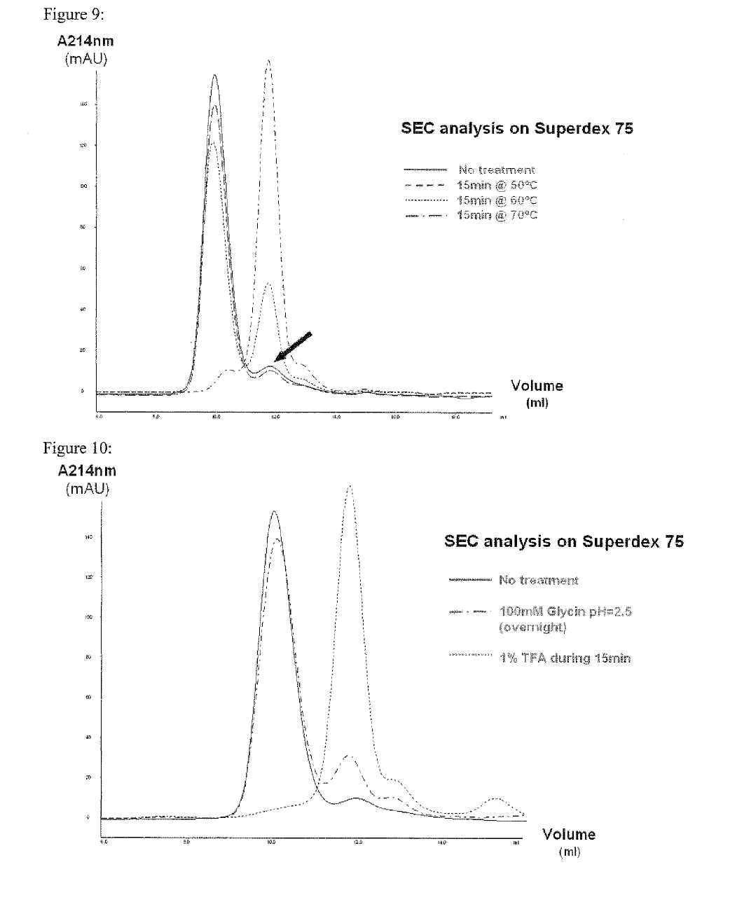

The Polypeptide A NFD was generated during a Polypeptide A preparation (see above) and was stored at −20° C. for 2.5 years. This dimeric material was obtained via Protein A chromatography and Size Exclusion Chromatography (SEC) in PBS. In the latter, monomeric and dimeric material were separated to a preparation of >95% pure dimer. Upon thawing about 5% monomeric material was detected (see arrow in FIG. 9). The concentration of dimeric material was 0.68 mg/ml.

### Analytic Size Exclusion Chromatography

The stability of the Polypeptide A NFD dimer was analysed via analytic SEC on a Superdex 75 10/300GL column (17-5174-01, GE Healthcare) using an Äkta Purifier10 workstation (GE Healthcare). The column was equilibrated in D-PBS at room temperature (20° C.). A flow rate of 1 ml/min was used. Proteins were detected via absorption at 214 nm. 12 μg samples of Polypeptide A NFD were injected.

Overview Analytic SEC Runs:
20 μl POLYPEPTIDE A NFD+90 μl D-PBS→15′/50° C.→100 μl analyzed
20 μl POLYPEPTIDE A NFD+90 μl D-PBS→15′/20° C.→100 μl analyzed
20 μl POLYPEPTIDE A NFD+90 μl D-PBS→30′/45° C.→100 μl analyzed
20 μl POLYPEPTIDE A NFD+90 μl D-PBS→15′/60° C.→100 μl analyzed
20 μl POLYPEPTIDE A NFD+90 μl D-PBS→15′/70° C.→100 μl analyzed
20 μl POLYPEPTIDE A NFD+90 μl [100 mM piperazin pH=10.2]→ON/4° C.→100 μl analyzed
20 μl POLYPEPTIDE A NFD+90 μl [100 mM Glycin pH=2.5]→ON/4° C.→100 μl analyzed
20 μl POLYPEPTIDE A NFD+90 μl [10% Isopropanol]→ON/4° C.→100 μl analyzed
20 μl POLYPEPTIDE A NFD+90 μl [30% Isopropanol]→ON/4° C.→100 μl analyzed
20 μl POLYPEPTIDE A NFD+90 μl [1% TFA]→15′/20° C.→100 μl analyzed
20 μl POLYPEPTIDE A NFD+90 μl [30% Isopropanol]→15′/50° C.→100 μl analyzed
20 μl POLYPEPTIDE A NFD+90 μl [30% Isopropanol]→15′/20° C.→100 μl analyzed
20 μl POLYPEPTIDE A NFD+90 μl [30% Isopropanol]→15′/40° C.→100 μl analyzed
20 μl POLYPEPTIDE A NFD+90 μl [30% Isopropanol]→15′/45° C.→100 μl analyzed

This material was used in several experiments: 20 μl dimer fractions were diluted with 90 μl D-PBS or other solvents, incubated under different conditions and 100 μl samples were analysed via analytic SEC.

### Tests:

In a first set of experiments incubation during 15 minutes at increasing temperatures was carried out (45, 50, 60 and 70° C.), followed by analytic SEC (Superdex 75™ 10/300GL). An incubation at 70° C. during 15 min resulted in an almost complete shift to monomeric Polypeptide A, whereas lower temperatures (e.g. 50° C.) did not result in such a drastic effect. After 15 minutes at 60° C. about 25% dissociated material was detected (see FIG. 9).

In a second set of experiments the effect of pH on the stability of Polypeptide A NFD was explored. 20 μl NFD was mixed with 90 μl [100 mM piperazin pH=10.2] or 90 μl [100 mM Glycine, pH=2.5] and incubated overnight (ON) at 4° C. 20 μl NFD was mixed with 90 μl [1% TFA] at room temperature for 15 minutes and then immediately analysed via SEC. The control was incubated in D-PBS. Samples were analysed via SEC the next day (see FIG. 10).

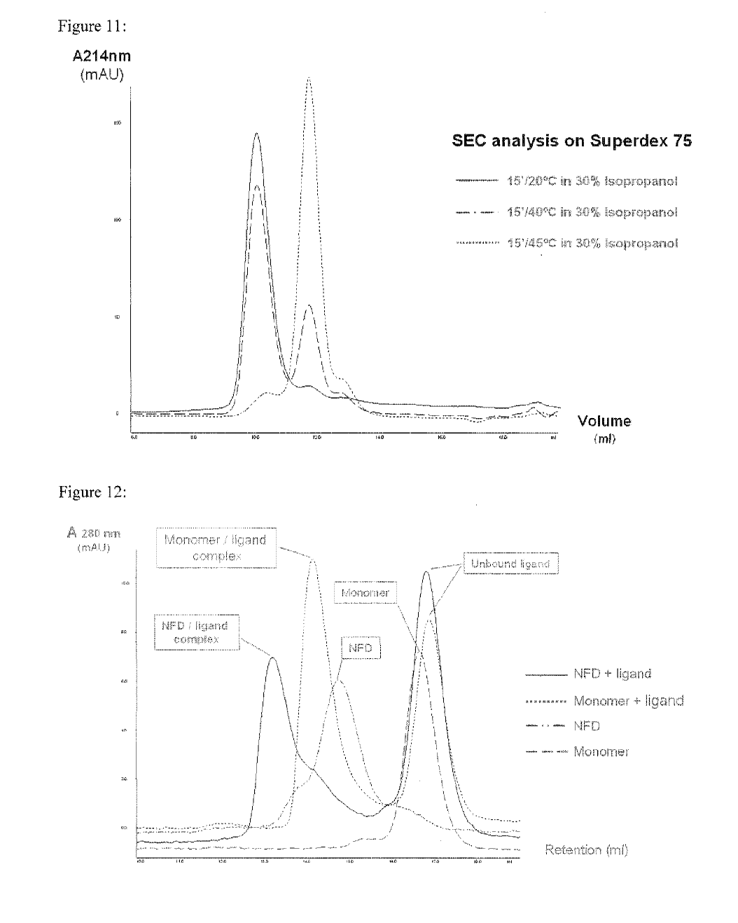

A third set of experiments consisted of a combined treatment: Temperature and organic solvent (Isopropanol). Neither incubation in 10 or 30% Isopropanol overnight at 4° C., nor incubation in 10 or 30% Isopropanol during 15 minutes at room temperature resulted in any significant dissociation. However, combining increased temperatures and organic solvent resulted in a much faster dissociation into monomer. Whereas incubation at 45° C. or 30% Isopropanol had no effect alone, combining both (during 15 minutes) resulted in an almost full dissociation into monomer. Isopropanol treatment at 40° C. yielded only 30% dissociation (see FIG. 11).

### Discussion

The concentration independent character of the dimer/monomer equilibrium was further substantiated by the near irreversibility of the interaction under physiological conditions. In addition, the rather drastic measures that need to be applied to (partly) dissociate the dimer into monomer point to an intrinsic strong interaction. Dissociation is only obtained by changing the conditions drastically (e.g. applying a pH below 2.0) or subjecting the molecule to high energy conditions. Temperature stability studies (data not shown) indicate that the Tm of Polypeptide A NFD is 73° C., so the observed dissociation into monomer might be indeed linked to (partial) unfolding.

The solubilizing properties of TFA combined with protonation at extreme low pH, increasing the hydrophilicity, also results in dissociation.

The combination of elevated temperature and organic solvent dissociation indicates that the interaction is mainly based on e.g. hydrophobicity (e.g. Van der Waals force), hydrogen bonds, and/or ionic interactions.

The conditions used to drive these dimers apart may be also useful to explore when determining further methods for producing these dimers, i.e. combining these procedures (e.g. temperature of higher than 75 degrees Celsius) with a high polypeptide concentration.

### Example 3

### Ligand Binding of NFDs

### Study of Ligand A (SEQ ID NO: 6) Binding to Polypeptide A and Polypeptide A NFD-Di Via Analytic Size Exclusion

### Ligand A Production:

Ligand A is known to be the binding domain of Polypeptide A, i.e. comprises the epitope of Polypeptide A (i.e. Ligand A represents the A1 domain of vWF).

Ligand A [1.46 mg/ml] was produced via Pichia in shaker flasks. Biomass was produced in BGCM medium. For induction a standard medium switch to methanol containing medium (BMCM) was done. The secreted protein was captured from the medium via IMAC, further purified on a Heparin affinity column and finally formulated in 350 mM NaCl in 50 mM Hepes via Size Exclusion Chromatography (SEC) (Superdex 75 HiLoad 26/60).

### Analytic SEC on Superdex 200 10/300GL (FIG. 12):

Polypeptide A (with 2 expected binding sites) and its corresponding NFD (with 4 expected binding sites) were obtained as disclosed in example 1 and added to 5× excess of the Ligand A (SEQ ID NO: 1). The resulting shift in molecular weight was studied via size exclusion chromatography (SEC). The shift in retention approximately indicates the number of Ligand A molecules binding to the Polypeptide A or corresponding NFD. Ligand A has a molecular weight of about 20 kDa. The molecular weight shift of the NFD/Ligand A complex compared to NFD alone or Polypeptide/Ligand A complex to Polypeptide A indicates the number of Ligand A per NFD or per Polypeptide A bound (see Table 2).

### Overview Analytic SEC Runs on Superdex 75 10/300GL

(B7)040308.1: Complex ligand-NFD 5 μl mix (ON stored at 4° C.)+80 μl A buffer
(B7)040308.2: 20 μl Molecular weight marker+80 μl A
(B7)040308.3: Complex 20 μl ligand+90 μl A, 4 h at RT+Polypeptide A [17 μl 1/10], 30 min at RT before analysis

### (B7)040308.4: Polypeptide A [17 μl in 90 μl A]

(B7)040308.5: Ligand in A buffer (1 h at RT)+Polypeptide A, 15 min at RT before analysis.

### (B7)040308.6: Ligand+Buffer A+NFD

(B7)040308.7: rest sample #6 after 1 h at RT

### (B7)040308.8: Buffer A+NFD

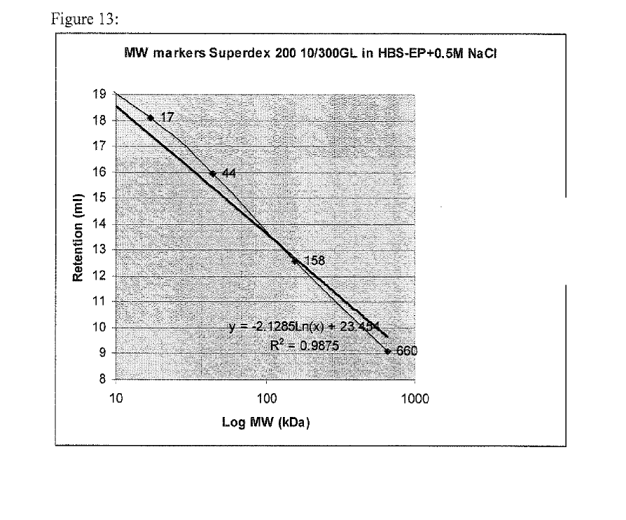

TABLE 2
*MW was calculated based on curve fitting of Molecular weight standards (Biorad
#151-1901) run on the same column under same conditions (see FIG. 13).

| | | | | Measured | Estimated |
|---|---|---|---|---|---|
| | | Measured | | MW shift with | Number |
| | Retention | MW | Theoretical MW | ligand A | of Ligand |
| Material | (ml) | (KDa)* | (Da) | exposure | A bound |
| NFD + Ligand A | 13.2 | 123.6 | 153940 (assuming | 62.5 | 3 |
| | | | 4 Ligand A | | |
| | | | bindings) | | |
| Polypeptide A + | 14.1 | 79.1 | 76970 (assuming | 54.1 | 2 |
| ligand A | | | 2 Ligand A | | |
| | | | bindings) | | |
| NFD | 14.7 | 61.1 | (55752) | Not | Not |
| | | | | applicable | applicable |
| Polypeptide A | 16.6 | 25.0 | (27876) | Not | Not |
| | | | | applicable | applicable |
| Ligand A | 16.8 | 22.8 | (24547) | Not | Not |
| | | | | applicable | applicable |

The correlation of the expected MW shows that more than 2 ligands (likely 3 and possibly 4 due to the atypical behaviour of Ligand A complexes on the SEC) are bound by the NFD.

### Example 4

### Further Characterization of a NFD with Polypeptide B

### Example 4.1

### Crystal Structure of a Non-Fused Dimer: Polypeptide B

### Crystallization

The protein was first concentrated to a concentration of about 30 mg/mL. The purified protein was used in crystallization trials with approximately 1200 different conditions. Conditions initially obtained have been optimized using standard strategies, systematically varying parameters critically influencing crystallization, such as temperature, protein concentration, drop ratio and others. These conditions were also refined by systematically varying pH or precipitant concentrations.

### Data Collection and Processing

Crystals have been flash-frozen and measured at a temperature of 100K. The X-ray diffraction data have been collected from the crystals at the SWISS LIGHT SOURCE (SLS, Villingen. Switzerland) using cryogenic conditions.

The crystals belong to the space group P 2₁ with 2 molecules in the asymmetric unit. Data were processed using the program XDS and XSCALE. Data collection statistics are summarized in Table 3.

TABLE 1
Statistics of data collection and processing

| X-ray source | PX-3 (SLS¹) |
|---|---|
| Wavelength (Å) | 0.97800 |
| Detector | MARCCD |
| Temperature (K) | 100 |
| Space group | P 2₁ |
| Cell dimensions: | |
| a; b; c (Å) | 37.00; 670.6; 41.14 |
| α; β; γ (°) | 90.0; 97.7; 90.0 |
| Resolution (Å)² | 1.20 (1.30-1.26) |
| Unique reflections² | 60716 (4632) |
| Multiplicity² | 4.1 (4.1) |
| Completeness (%)² | 97.7 (96.7) |
| R_{sym} (%)^{2,3} | 7.2 (41.4) |
| Rₘₑₐₛ (%)^{2,4} | 8.3 (47.6) |
| I/σ² | — (—) |
| Mean(I)/sigma^{2,5} | 12.83 (4.01) |

¹SWISS LIGHT SOURCE (SLS, Villingen, Switzerland)
²Numbers in brackets corresponds to the resolution bin with R_{sym} = 41.4%
${ }^{3} {R}_{sym} = \frac{{\sum }_{h} {\sum }_{i}^{{n}_{h}} \vert {\hat{I}}_{h} - {I}_{h , i} \vert}{{\sum }_{h} {\sum }_{i}^{{n}_{h}} {I}_{h , i}}$ $with {\hat{I}}_{h} = \frac{1}{n} {\sum }_{i}^{{n}_{h}} {I}_{h , i} , where {I}_{h , i} is the intensity value of the ith measurement of h$ ${ }^{4} {R}_{sym} = \frac{{\sum }_{h} \sqrt{\frac{{n}_{h}}{{n}_{h} - 1}} {\sum }_{i}^{{n}_{h}} \vert {\hat{I}}_{h} - {I}_{h , i} \vert}{{\sum }_{h} {\sum }_{i}^{{n}_{h}} {I}_{h , i}}$ $with {\hat{I}}_{h} = \frac{1}{n} {\sum }_{i}^{{n}_{h}} {I}_{h , i} , where {I}_{h , i} is the intensity value of the ith measurement of h$ ⁵Calculated from independent reflections

### Structure Modelling and Refinement

The phase information necessary to determine and analyze the structure was obtained by molecular replacement.

Subsequent model building and refinement was performed according to standard protocols with the software packages CCP4 and COOT. For the calculation of the R-factor, a measure to cross-validate the correctness of the final model, 1.6% of measured reflections were excluded from the refinement procedure (Table 4).

The ligand parameterisation was carried out with the program CHEMSKETCH. LIBCHECK (CCP4) was used for generation of the corresponding library files.

Statistics of the final structure and the refinement process are listed in Table 4.

TABLE 4
refinement statistics¹

| | |
|---|---|
| Resolution (Å) | 20.0-1.20 |
| Number of reflections | 59743/972 |
| (working/test) | |
| R_{cryst} (%) | 14.8 |
| R_{free} (%) | 16.9 |
| Total number of atoms in protein | 1759 |
| Deviation from ideal geometry² | |
| Bond lengths (Å) | 0.006 |
| Bond angles (°) | 1.17 |

¹Values as defined in REFMAC5, without sigma cut-off
²Root mean square deviations from geometric target values

### Overall Structure

The asymmetric unit of crystals is comprised of 2 monomers. The nanobody is well resolved by electron density maps.

### Structure

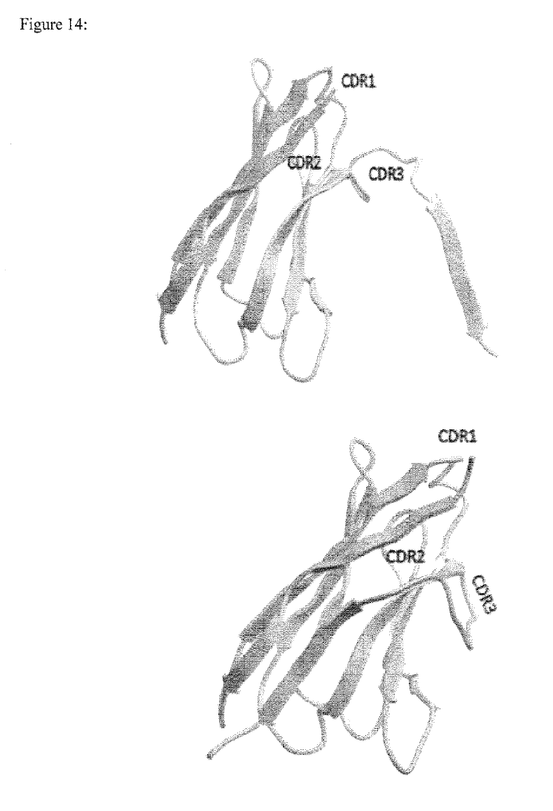

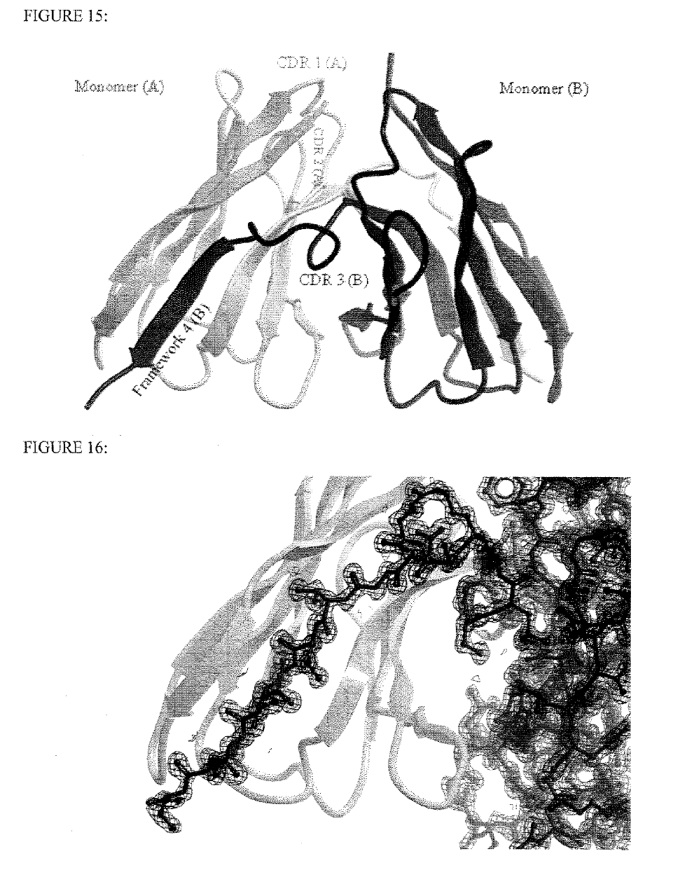

The 2 polypeptide B-monomers that form the polypeptide B dimer (NFD-Mo) have a properly folded CDR1 and CDR2 and framework 1-3. The framework 4 residues (residues 103-113 according to the Kabat numbering scheme) are exchanged between the 2 monomers. This results in an unfolded CDR3 of both monomers that are present in the dimer (see FIG. 14). Dimer formation is mediated by the exchange of a β-strand from Q105 to Ser113 between both monomers (see FIG. 15). Strand exchange is completely defined by electron density (see FIG. 16).

The residues of framework 1-3 and CDR1 & CDR2 of the monomer that form the dimer have a classical VHH fold and are almost perfectly superimposable on a correctly folded polypeptide B VHH domain (backbone rmsd<0.6 Å). A decreased stabilization of CDR3 in polypeptide B compared to the structures of VHH's with similar sequences to polypeptide B can be one of the causes of the framework 4 exchanged dimerization. A slightly modified form of polypeptide B with a Proline at position 45 shows a hydrogen-bond between Y91 and the main-chain of L98. This hydrogen-bond has a stabilizing effect on the CDR3 conformation.

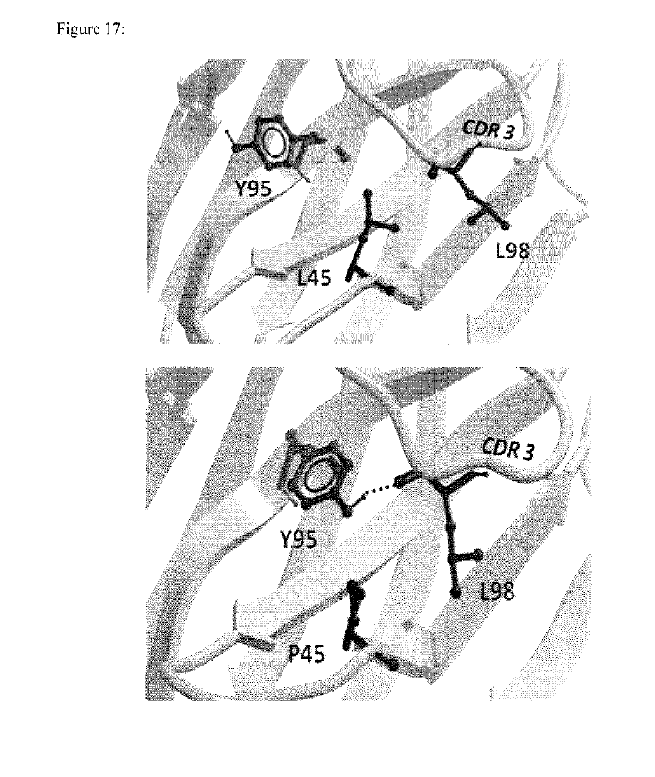

Due to the leucine at position 45 in polypeptide B, the tyrosine 91 can not longer form the hydrogen-bond with the main-chain of leucine-98. This leads to a decreased stabilization of the CDR3 conformation in polypeptide B (FIG. 17).

### Example 4.2

### Stability and Various Other Studies of the NFD with Polypeptide B

### Production and Isolation of Polypeptide B

Tagless polypeptide B was over-expressed in E. coli TOP10 strain at 28° C. after overnight induction with 1 mM IPTG. After harvesting, the cultures were centrifuged for 30 minutes at 4500 rpm and cell pellets were frozen at −20° C. Afterward the pellets were thawed and re-suspended in 50 mM phosphate buffer containing 300 mM NaCl and shaken for 2 hours at room temperature. The suspension was centrifuged at 4500 rpm for 60 minutes to clear the cell debris from the extract. The supernatant containing polypeptide B, was subsequently loaded on Poros MabCapture A column mounted on Akta chromatographic system. After washing the affinity column extensively with D-PBS, bound polypeptide B protein was eluted with 100 mM Glycine pH 2.7 buffer. Fractions eluted from column with acid were immediately neutralized by adding 1.5M TRIS pH 8.5 buffer. At this stage the protein is already very pure as only a single band of the expected molecular weight is observed on Coomassie-stained SDS-PAGE gels. The fractions containing the polypeptide B were pooled and subsequently concentrated by ultrafiltration on a stirred cell with a polyethersulphone membrane with a cut-off of 5 kDa (Millipore). The concentrated protein solution was afterwards loaded on a Superdex 75 XK 26/60 column. On the chromatogram (see figure X), besides the main peak eluting between 210 mL and 240 mL, a minor peak eluting between 180 mL and 195 ml was present.

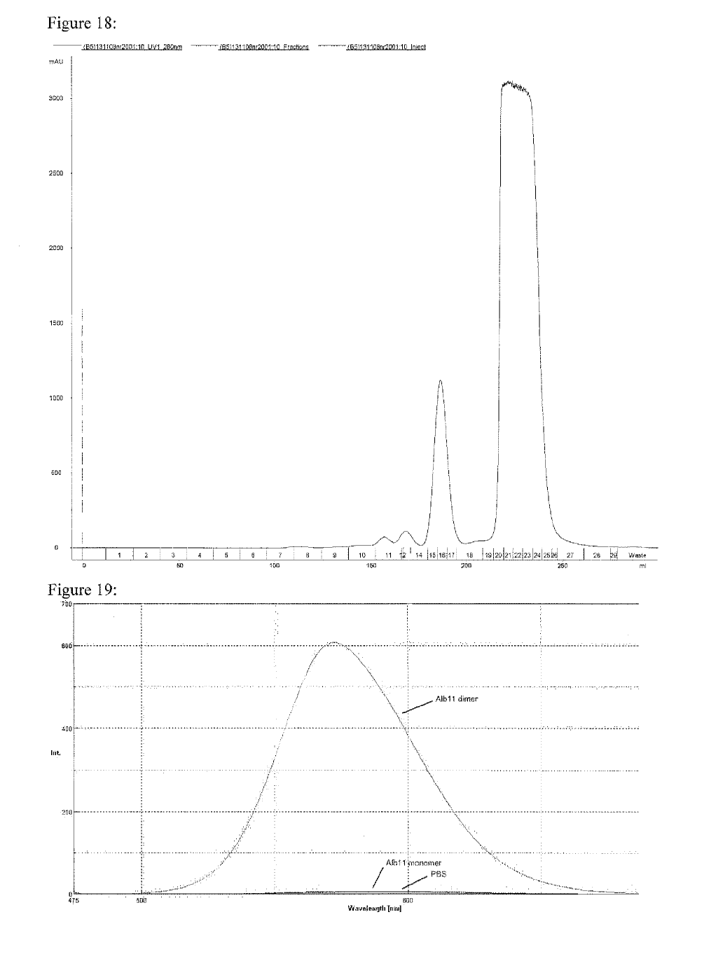

Analysis on SDS-PAGE uncovered that both major peaks contain a single polypeptide with the same mobility (FIG. 18). This observation was the first indication that the peak eluting between 180 mL and 195 mL is a dimeric species, whereas the material eluting between 210 mL and 240 mL is a monomer. Further analysis on reversed phase chromatography and LC/MS of the dimeric and monomer species uncovered that both contain the same polypeptide with a molecular weight of about 12110 dalton. In this way from a 10 L fermentor run, in total 30 mg of the dimeric species and 1200 mg of the monomeric form of polypeptide B was isolated.

### Antigen Binding Properties

The binding of the polypeptide B monomer and Polypeptide B dimer to human serum albumin was tested by surface plasmon resonance in a Biacore 3000 instrument. In these experiments human serum albumin was immobilized on CM5 chip via standard amine coupling method. The binding of both monomeric polypeptide B and dimeric polypeptide B at a concentration of 10 nanomolar were tested. Only for the monomer, binding was observed whereas no increase in signal was observed for the dimeric polypeptide B.

### Difference in Physicochemical Properties Between Monomeric and Dimeric Polypeptide B

The fluorescent dye Sypro orange (5000× Molecular Probes) can be used to monitor the thermal unfolding of proteins or to detect the presence of hydrophobic patches on proteins. In the experiment, monomeric and dimeric Polypeptide B at a concentration of 150 microgram/mL were mixed with Sypro orange (final concentration 10×). The solution was afterwards transferred to quartz cuvette, and fluorescence spectra were recorded on A Jasco FP6500 instrument. Excitation was at 465 nm whereas the emission was monitored from 475 to 700 nm. As shown in FIG. 19, only a strong signal for the dimeric polypeptide B, whereas the no increase in fluorescence emission intensity was observed for the polypeptide B monmeric species. This observation strongly suggests that monomeric and dimeric forms of polypeptide B have a distinct conformation.

### AUC-EQ—Sedimentation-Diffusion Equilibrium

### Material and Methods

Experiments were performed with an Analytical ultracentrifuge XL-I from Beckman-Coulter using the interference optics of the instrument. Data were collected at a temperature of 20° C. and rotational speeds of 25000 rpm and 40000 rpm. 150 μL were filled in the sample sector of 12 mm two sector titanium centerpieces. Samples were diluted with standard PBS, which was also used for optical referencing. Attainment of apparent chemical and sedimentation equilibrium was verified by comparing consecutive scans until no change in concentration with time was observed. Data were evaluated with the model-independent M*-function and various explicit models using NONLIN. Standard values for the ν̅ of the protein and the density of the solvent were used. Where appropriate, 95% confidence limits are given in brackets.

### Result

Polypeptide B is found to have a molar mass of 11.92 kg/mole (11.86-11.97) kg/mole from a fit assuming a single, monodispere component. This agrees well with the result from the model-free analysis which is 12.25 kg/mole at zero concentration. Attempts to describe the data assuming self-association, non-ideality or polydispersity did not improve the global rmsd of the fit.

Polypeptide B is equally well-defined, having a molar mass of 23.06 kg/mole (22.56-23.44) kg/mole based on a direct fit assuming a single, monodispere component. The model-free analysis reveals a molar mass of 22.69 kg/mole. A small contribution from thermodynamic non-ideality improved the fit slightly but did not alter the molar mass.

No evidence for a reversible self-association could be found.

The ratio of the M(Polypeptide B-dimer)/M(Polypeptide B) is 1.93. The small deviation from the expected factor of 2 can be explained by a different ν̅ of Polypeptide B Dimer compared to Polypeptide B, slight density differences for the different dilutions due to the slightly different Polypeptide B, slight density differences for the dilutions due to the slightly different buffers used (PBS for dilution and D-PBS for the stock solutions) and a contribution from non ideality too small to be reliably described with the data available.

### Stability Study of Polypeptide F and Polypeptide B at 4° C., 25° C. and 37° C.

Solutions of monomeric polypeptide F and polypeptide B, formulated in D-PBS, were concentrated to 20 mg/mL and put on storage at 4° C. 25° C. and 37° C. After 3 and 6 weeks samples were analyzed by size exclusion chromatography on a Phenomenex BioSep SEC S-2000 column. In the SEC chromatograms of both polypeptide F and Polypeptide B, the presence of a pre-peak was only observed in the chromatograms of the samples stored at 37° C. The pre-peak corresponding to a dimer, was not observed in samples stored at 4° C., 25° C. or in a reference material stored at −20° C.

In the table 5 below the percentage of dimer present in the samples stored at 37° C. (expressed as percentage of area of dimer versus total area) for both polypeptide F and polypeptide B are compiled. As can be observed in this table, it appears that polypeptide B is more susceptible to dimer formation than polypeptide F.

TABLE 5

| Nanobody | % dimer-3 weeks | % dimer-6 weeks |
|---|---|---|
| Polypeptide F | 3.1 | 5.8 |
| Polypeptide B | 20.9 | 37.1 |

In a separate experiment the effect of mannitol as excipient in the formulation buffer was evaluated. In this case monomeric polypeptide B was formulated at a protein concentration of 18 mg/mL respectively in D-PBS or D-PBS containing 5% mannitol. Samples were stored at 37° C. and analyzed by size exclusion chromatography on a Phenomenex BioSep SEC S-2000 column after 2, 4, 6 and 8 weeks.

In the table 6 below, the percentage of dimer present in the samples stored at 37° C. (expressed as percentage of area of dimer versus total area) for Polypeptide B stored in D-PBS and in D-PBS/5% mannitol were compiled. As shown is this table, the presence of mannitol in the buffer has a clear effect on the kinetics of dimer formation of polypeptide B at 37° C.

TABLE 6

| | % dimer after | % dimer after | % dimer after | % dimer after |
|---|---|---|---|---|
| | 2 weeks | 4 weeks | 6 weeks | 8 weeks |
| Polypeptide B | 13.5 | 22.1 | 30.0 | 41.8 |
| Polypeptide B | 5.3 | 11.7 | 16.8 | 23.7 |
| with 5% | | | | |
| mannitol | | | | |

In another experiment, solutions of both monomeric polypeptide F and polypeptide B at concentrations of 5 mg/ml, 10 mg/mL and 20 mg/mL in D-PBS were stored at 37° C. After 6 weeks, samples were analyzed by size exclusion chromatography on a Phenomenex BioSep SEC S-2000 column. In the table below the percentage of dimer present in the samples stored at 37° C. (expressed as percentage of area of dimer versus total area) for polypeptide F and polypeptide B stored at 5 mg/mL, 10 mg/mL and 20 mg/mL are compiled. From this experiment we learned as observed earlier that dimer formation proceeds faster for the polypeptide B than for polypeptide F, but also that the kinetics of dimer formation are largely dependent on the protein concentration.

TABLE 7

| | % dimer | % dimer | % dimer |
|---|---|---|---|
| | (5 mg/mL) | (10 mg/mL) | (20 mg/mL) |
| Polypeptide F | 1.2 | 3.1 | 5.7 |
| Polypeptide B | 13.0 | 20.6 | 36.9 |

Similarly, dimer and possibly multimer formation was observed for polypeptides comprising polypeptide B and other single variable domains, e.g. polypeptides comprising one polypeptide N and 2 nanobodies binding to a therapeutic target (e.g. 2 identical nanobody directed against a therapeutic target). The dimer/multimer formation of said polypeptides comprising e.g. polypeptide B and other Nanobodies could be slowed down or in some instances almost avoided if they were formulated in a mannitol containing liquid formulation. Other polyols and/or sugars that are believed to be beneficial to reduce or avoid the formation of dimers (NFDs) and other possibly higher multimers are listed in Table 8. A wide variety of liquid formulations may be useful which may consist of any buffering agent, a biologically effective amount of polypeptide of the invention, a concentration of mannitol that is no greater than approximately 0.6M and other excipients including polyols, non-reducing sugars, NaCl or amino acids.

TABLE 8

| | |
|---|---|
| Polyols | sorbitol, mannitol, xylitol, ribitol, erythritol |
| Non-reducing sugars | sucrose, trehalose |

### Chaotrope Induced Unfolding of Polypeptide B and Polypeptide B Dimer

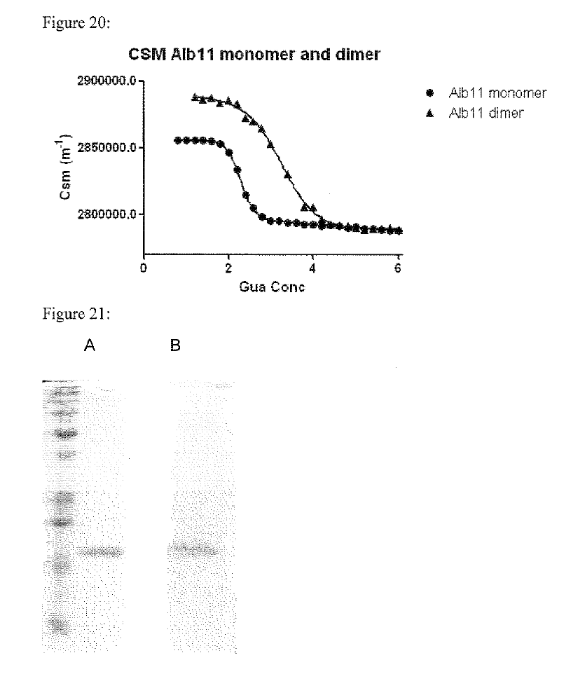

Chaotrope induced unfolding is a technique frequently used to assess the stability of proteins. To monitor chaotrope induced unfolding intrinsic fluorescence of tryptophan or tyrosine residue can be used. As unfolding parameter the ‘center of spectral mass’ (CSM=Σ(fluorescence intensity×wavenumber)/Σ(fluorescence intensity) can be used. Unfolding experiments with Polypeptide B monomer and Polypeptide B dimer were performed at 25 μg/mL in guanidine solution in the concentration range 0-6M. After overnight incubation of these solutions fluorescence spectra were recorded using a Jasco FP-6500 instrument. Excitation was at 295 nm and spectra were recorded between 310 to 440 nm. Using the spectral data the CSM-value was calculated using the formula above. In the FIG. 20, the CSM as a function of guanidine concentration is shown. As can be observed in FIG. 20, polypeptide B (=Alb11) dimer unfolds at higher concentrations of guanidine, and allows us to conclude that the monomer is less stable than the Polypeptide B-dimer.

### Example 5

### Further Characterization of a NFD with Polypeptide G and H

Different mutants of polypeptide F have been constructed, expressed and purified. Sequence information is provided below.

Purity was analysed on a Coomassie stained gel (FIG. 21) and western blot.

### Binding to Serum Albumin in Biacore

Binding of Nanobodies to human serum albumin (HSA) is characterized by surface plasmon resonance in a Biacore 3000 instrument, and an equilibrium constant K_{D} is determined. In brief, HSA was covalently bound to CM5 sensor chips surface via amine coupling until an increase of 500 response units was reached. Remaining reactive groups were inactivated. Nanobody binding was assessed using series of different concentrations. Each Nanobody™ concentration was injected for 4 min at a flow rate of 45 μl/min to allow for binding to chip-bound antigen. Next, binding buffer without Nanobody was sent over the chip at the same flow rate to allow dissociation of bound Nanobody. After 15 minutes, remaining bound analyte was removed by injection of the regeneration solution (50 mM NaOH).

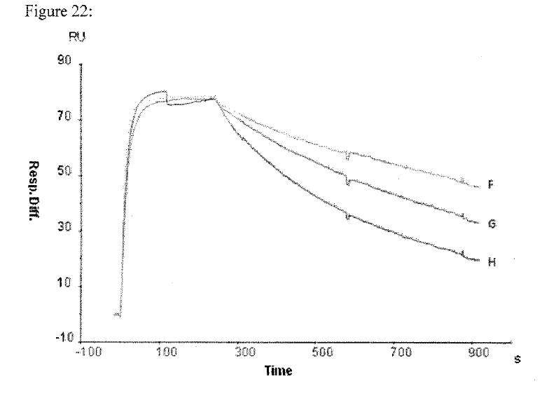

From the sensorgrams obtained (FIG. 22) for the different concentrations of each analyte. K_{D} values were calculated via kinetic data analysis. Polypeptide H (with introduction of GL instead of EP, in particular P is replaced by L, see also FIG. 17 and examples above) has a greater koff rate.

TABLE 9
k_{off} values of Polypeptide F and the humanized
derivatives Polypeptide G and Polypeptide H
as determined in Biacore for binding to HSA.

| Nanobody | K_{off} (1/s) |
|---|---|
| Polypeptide F | 6.83E−4 |
| Polypeptide G | 1.18E−3 |
| Polypeptide H | 1.97E−3 |

### Stability on Storage

Solutions of monomeric Polypeptide G and Polypeptide H, formulated in D-PBS, are concentrated to 20 mg/mL and put on storage at 4° C., 25° C. and 37° C. After 3 and 6 weeks samples are analyzed by size exclusion chromatography on a Phenomenex BioSep SEC S-2000 column.

TABLE A
Sequence Listings:

| | SEQ | |
|---|---|---|
| | ID | |
| Code | NO: | Sequence |
| Polypeptide A | 1 | EVQLVESGGGLVQPGGSLRLSCAASGRIFSYNPMGWERQAPGKGR |
| | | ELVAAISRTGGSTYYPDSVEGRFTISRDNAKRMVYLQMNSLRAEDT |
| | | AVYYCAAAGVRAEDGRVRTLPSEYTFWGQGTQVTVSSAAAEVQL |
| | | VESGGGLVQPGGSLRLSCAASGRTFSYNPMGWFRQAPGKGRELVA |
| | | AISRTGGSTYYPDSVEGRFTISRDNAKRMVYLQMNSLRAEDTAVY |
| | | YCAAAGVRAEDGRVRTLPSEYTFWGQGTQVTVSS |
| Polypeptide B | 2 | EVQLVESGGGLVQPGNSLRLSCAASGFTFSSFGMSWVRQAPGKGL |
| | | EWVSSISGSGSDTLYADSVKGRFTISRDNAKTTLYLQMNSLRPEDT |
| | | AVYYCTIGGSLSRSSQGTLVTVSS |
| Polypeptide C | 3 | EVQLVESGGGLVQPGGSLRLSCAASGFTFSDYDIGWERQAPGKGR |
| | | EGVSGISSSDGNTYYADSVKGRFTISRDNAKNTLYLQMNSLRPEDT |
| | | AVYYCAAEPPDSSWYLDGSPEFFKYWGQGTLVTVSSGGGGSGGGS |
| | | EVQLVESGGGLVQPGNSLRLSCAASGFTFSSFGMSWVRQAPGKGL |
| | | EWVSSISGSGSDTLYADSVKGRFTISRDNAKTTLYLQMNSLRPEDT |
| | | AVYYCTIGGSLSRSSQGTLVTVSSGGGGSGGGSEVQLVESGGGLVQ |
| | | PGGSLRLSCAASGFTFSDYDIGWFRQAPGKGREGVSGISSSDGNTY |
| | | YADSVKGRFTISRDNAKNTLYLQMNSLRPEDTAVYYCAAEPPDSS |
| | | WYLDGSPEFFKYWGQGTLVTVSS |
| Polypeptide D | 4 | EVQLVESGGGLVQPGGSLRLSCAASGFTFSDYDIGWFRQAPGKGR |
| | | EGVSGISSSDGNTYYADSVKGRFTISRDNAKNTLYLQMNSLRPEDT |
| | | AVYYCAAEPPDSSWYLDGSPEFFKYWGQGTLVTVSSDAHKSEVA |
| | | HRFKDLGEENFKALVLIAFAQYLQQCPFEDHVKLVNEVTEFAKTC |
| | | VADESAENCDKSLHTLFGDKLCTVATLRETYGEMADCCAKQEPER |
| | | NECFLQHKDDNPNLPRLVRPEVDVMCTAFHDNEETFLKKYLYEIA |
| | | RRHPYFYAPELLFFAKRYKAAFTECCQAADKAACLLPKLDELRDE |
| | | GKASSAKQRLKCASLQKFGERAFKAWAVARLSQRFPKAEFAEVSK |
| | | LVTDLTKVHTECCHGDLLECADDRADLAKYICENQDSISSKLKECC |
| | | EKPLLEKSHCIAEVENDEMPADLPSLAADFVESKDVCKNYAEAKD |
| | | VFLGMFLYEYARRHPDYSVVLLLRLAKTYETTLEKCCAAADPHEC |
| | | YAKVFDEFKPLVEEPQNLIKQNCELFEQLGEYKFQNALLVRYTKK |
| | | VPQVSTPTLVEVSRNLGKVGSKCCKHPEAKRMPCAEDYLSVVLNQ |
| | | LCVLHEKTPVSDRVTKCCTESLVNRRPCFSALEVDETYVPKEFNAE |
| | | TFTFHADICTLSEKERQIKKQTALVELVKHKPKATKEQLKAVMDDF |
| | | AAFVEKCCKADDKETCFAEEGKKLVAASQAALGL |
| Polypeptide E | 5 | EVQLVESGGGLVQPGGSLRLSCAASGFTFSDYDIGWFRQAPGKGR |
| | | EGVSGISSSDGNTYYADSVKGRFTISRDNAKNTLYLQMNSLRPEDT |
| | | AVYYCAAEPPDSSWYLDGSPEFFKYWGQGTLVTVSSGGGGSGGGS |
| | | EVQLVESGGGLVQPGNSLRLSCAASGFTFSSFGMSWVRQAPGKGL |
| | | EWVSSISGSGSDTLYADSVKGRFTISRDNAKTTLYLQMNSLRPEDT |
| | | AVYYCTIGGSLSRSSQGTLVTVSS |
| Polypeptide F | 6 | AVQLVESGGGLVQPGNSLRLSCAASGFTFRSFGMSWVRQAPGKEP |
| | | EWVSSISGSGSDTLYADSVKGRFTISRDNAKTTLYLQMNSLKPEDT |
| | | AVYYCTIGGSLSRSSQGTQVTVSS |
| Ligand A | 7 | DISEPPLHDFYCSRLLDLVFLLDGSSRLSEAEFEVLKAFVVDMMER |
| | | LRISQKWVRVAVVEYHDGSHAYIGLKDRKRPSELRRIASQVKYAG |
| | | SQVASTSEVLKYTLFQIFSKIDRPEASRIALLLMASQEPQRMSRNFV |
| | | RYVQGLKKKKVIVIPVGIGPHANLKQIRLIEKQAPENKAFVLSSVDE |
| | | LEQQRDEIVSYLCDLAPEAPPPTHHHHHH |
| CDR3 and FR4 | 8 | GGSLSRSSQGTLVTVSS |
| of polypeptide B | | |
| Polypeptide G | 9 | EVQLVESGGGLVQPGGSLRLSCAASGFTFRSFGMSWVRQAPGKEP |
| | | EWVSSISGSGSDTLYADSVKGRFTISRDNAKTTLYLQMNSLKPEDT |
| | | AVYYCTIGGSLSRSSQGTQVTVSS |
| Polypeptide H | 10 | EVQLVESGGGLVQPGGSLRLSCAASGFTFRSFGMSWVRQAPGKGL |
| | | EWVSSISGSGSDTLYADSVKGRFTISRDNAKTTLYLQMNSLKPEDT |
| | | AVYYCTIGGSLSRSSQGTQVTVSS |

The terms and expressions which have been employed are used as terms of description and not of limitation, and there is no intention in the use of such terms and expressions of excluding any equivalents of the features shown and described or portions thereof, it being recognized that various modifications are possible within the scope of the invention.

All of the references described herein are incorporated by reference, in particular for the teaching that is referenced hereinabove.

### PREFERRED ASPECTS

1. A stable NFD.
2. A stable NFD in solution.
3. A stable NFD obtainable by a process comprising the step of concentrating a polypeptide comprising at least one single variable domain and/or by a process comprising the step of storage at elevated temperature, e.g. at a temperature close to the meting temperature or e.g. at 37° C. over a prolonged time period, e.g. such as 1 to 4 weeks, e.g. 4 weeks.
4. A stable NFD obtainable by a process comprising the step of concentrating a polypeptide consisting of single variable domain(s) and linkers.
5. A stable NFD according to the aspects 2 or 4, wherein the step of concentration is done by affinity- or ion exchange chromatography.
6. A stable NFD according to the aspects 2 to 5, wherein the step of concentration is done on a Protein A column and wherein high amounts of polypeptide are loaded on the column, e.g. 2 to 5 mg per ml resin Protein A.
7. A stable NFD according to the aspects 5 or 6, wherein the polypeptide is eluted by a steep pH gradient, e.g. a one step pH change of 2.
8. A stable NFD according to the previous aspects, wherein the NFD is stable over a period of up to 2 years at −20 degrees celcius.
9. A stable NFD according to the aspects above, wherein the NFD is stable over a period of up to 2 weeks at 4 degrees celcius.
10. A stable NFD according to the previous aspects, wherein the NFD is stable over a period of up to 15 minutes at 50 degrees celcius.
11. A stable NFD according to the previous aspects, wherein the NFD is stable at acidic pH.
12. A stable NFD according to the previous aspects, wherein the NFD is stable at acidic pH over prolonged period of time.
13. A stable NFD according to the previous aspects, wherein the NFD is stable at basic pH over a prolonged period of time.
14. A stable NFD according to the previous aspects, wherein the NFD is stable between pH 3 and pH 8.
15. A stable NFD according to the previous aspects, wherein the NFD is stable between pH 2.5 and pH 8.
16. A stable NFD according to the previous aspects, wherein the NFD is stable between pH 3 and pH 8 for 4 weeks at 4 degrees celcius.
17. A stable NFD according to the previous aspects, wherein the NFD is stable when mixing with organic solvents.
18. A stable NFD according to the previous aspects, wherein the NFD is stable when mixing with an alcohol, e.g. isopropanol.
19. A stable NFD according to the previous aspects, wherein the NFD is stable when mixing with 30% v/v of an alcohol, e.g. isopropanol.
20. A stable NFD according to the previous aspects, wherein the dissociation constant for the NFD to its target molecule is about the same as the dissociation constant for its corresponding monomeric building block to said target molecule.
21. A stable NFD according to the previous aspects, wherein there is no specific binding to its target molecule.
22. A stable NFD according to the previous aspects, wherein the dissociation constant for the NFD to its target molecule is 30% or less, preferably 20% or less, more preferably 10% or less, of the dissociation constant for its corresponding monomeric building block to said target molecule.
23. A stable NFD according to the previous aspects, wherein the dissociation constant for the NFD to its target molecule is 100 nM or less, preferably 10 nM or less, more preferably in M % or less.
24. A stable NFD according to the previous aspects, wherein the koff value for the NFD to its target molecule is about the same as the koff value of its corresponding monomeric building block.
25. A stable NFD according to the previous aspects, wherein the koff value for the NFD to its target molecule is not more than 90%, more preferably 50%, even more preferably 40%, even more preferably 30%, even more preferably 20%, most preferably 10% higher than the koff value of its corresponding monomeric building block.
26. A stable NFD according to the previous aspects, wherein the koff value for the NFD to its target molecule is not more than 50% higher than the koff value of its corresponding monomeric building block.
27. A stable NFD according to the previous aspects, wherein the koff value for the NFD to its target molecule is not more than 10% higher than the koff value of its corresponding monomeric building block.
28. A stable NFD according to the previous aspects, wherein the single variable domain is a Nanobody such as a VHH, a humanized VHH, an affinity-matured, stabilized or otherwise altered VHH or a construct thereof.
29. A stable NFD according to the previous aspects, wherein the single variable domain is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 9 and SEQ ID NO: 10, preferably SEQ ID NO: 2.
30. A stable NFD according to the previous aspects, wherein the single variable domain is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 9 and SEQ ID NO: 10, preferably SEQ ID NO: 2.
31. A stable NFD according to the previous aspects, wherein the single variable domain is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 9 and SEQ ID NO: 10, preferably SEQ ID NO: 2 and to a functional sequence that is at least 70%, more preferably 80%, even more preferably 90%, even more preferably 90%, most preferably 95% identical to any of SEQ ID NO: 1, SEQ ID NO: 2, SEQ ID NO: 3, SEQ ID NO: 4, SEQ ID NO: 5, SEQ ID NO: 6, SEQ ID NO: 9 and SEQ ID NO: 10, preferably SEQ ID NO: 2.
32. A stable NFD according to the previous aspects, wherein the single variable domain is selected from the group consisting of SEQ ID NO: 1, SEQ ID NO: 2, SEQ ID NO: 3, SEQ ID NO: 4, SEQ ID NO: 5, SEQ ID NO: 6. SEQ ID NO: 9 and SEQ ID NO: 10, preferably SEQ ID NO: 2; and to a functional sequence that is at least 70%, more preferably 80%, even more preferably 90%, even more preferably 90%, most preferably 95% identical to any of SEQ ID NO: 1, SEQ ID NO: 2, SEQ ID NO: 3, SEQ ID NO: 4, SEQ ID NO: 5, SEQ ID NO: 6, SEQ ID NO: 9 and SEQ ID NO: 10, preferably SEQ ID NO: 2; and wherein said sequences specifically bind to their target molecule(s), more preferably have a dissociation constant to at least one of their target molecules if bi- or multispecific, of 100 nM or less, even more preferably have a dissociation constant of 10 nM or less, most preferably have a dissociation constant of 1 nM or less.
33. A functional fragment of a NFD as described in aspects 1 to 32.
34. A polypeptide comprising at least one single variable domain; wherein said at least one of the single variable domains can form a NFD as e.g. described in aspects 1 to 32.
35. A preparation comprising a NFD as described in aspects 1 to 32, a functional fragment of aspect 33 or a polypeptide of aspect 34.
36. A preparation comprising a NFD as described in aspects 1 to 32, a functional fragment of aspect 33 or a polypeptide of aspect 34, wherein the ratio of NFD and its corresponding monomeric building block is about 1 part NFD to 1 part corresponding monomeric building block to about 1 part NFD to 2 parts corresponding monomeric building block.
37. A preparation comprising a NFD as described in aspects 1 to 32, a functional fragment of aspect 33 or a polypeptide of aspect 34, wherein the ratio of NFD and its corresponding monomeric building block is about 1 part NFD to 1 part corresponding monomeric building block to about 2 parts NFD to 1 part corresponding monomeric building block.
38. A preparation comprising a NFD as described in claims 1 to 32, a functional fragment of aspect 32 or a polypeptide of aspect 33, wherein the ratio of NFD and its corresponding monomeric building block is 25% NFD: 75% monomeric building block.
39. A preparation comprising a NFD as described in aspects 1 to 32, a functional fragment of aspect 33 or a polypeptide of aspect 34, wherein the ratio of NFD and its corresponding monomeric building block is 75% NFD: 25% monomeric building block.
40. A process of making a NFD according to aspects 1 to 32, a functional fragment of aspect 33 or a polypeptide of aspect 34 comprising the process step that has a condition that favors hydrophobic interactions.
41. A process of making a NFD according to aspect 40, wherein said process step is a purification step.
42. A process of making a NFD according to aspect 40, wherein within said process step, the condition is such that it promotes partial protein unfolding.
43. A process of making a NFD according to aspect 42, wherein said process step is a purification step.
44. A process of making a NFD comprising the step of up-concentrating the monomeric building blocks of said NFD e.g. by binding said polypeptides comprising single variable domain(s) on an affinity chromatography column, e.g. Protein A or IMAC.
45. A process of making a NFD comprising the step of binding polypeptides comprising single variable domain(s) on a affinity chromatography column, e.g. Protein A or IMAC, and eluting with a pH step which allows release of said polypeptide.
46. A process of making a NFD comprising the step of binding polypeptides comprising single variable domain(s) on a affinity chromatography column, e.g. Protein A, and eluting with a pH step which allows release of said polypeptide within 1 column volume.
47. A process of making a NFD comprising the step of ultra-filtration.
48. A process according to aspect 46 wherein the ultra-filtration is done under conditions of low salt.
49. A process of making a NFD according to aspects 1 to 32 comprising the process step of storing the appropriate polypeptide comprising at least a singe variable domain at elevated temperature over a prolonged time.
50. A process of making a NFD according to aspect 49, wherein said elevated temperature is 37° C. and time is 1, 2, 3, 4, 5, or 6, preferably 4 weeks.
51. A process of making a NFD according to aspect 49 to 50, wherein said elevated temperature is such that it promotes partial protein unfolding and exposure is over 1, 2, 3, 4, 5, or 6, preferably 4 weeks.
52. A process of making a NFD according to aspect 49 to 51, wherein said elevated temperature is close to the melting temperature of the polypeptide exposure is over 1, 2, 3, 4, 5, or 6, preferably 4 weeks.
53. A process of making a NFD according to aspect 48 to 52, wherein the CDR3 of said single variable domain is destabilized.
54. A process of making a NFD according to aspect 49 to 53, wherein said single variable domain is a Nanobody, such as e.g. a VHH, a humanized VHH, an affinity-matured, stabilized or otherwise altered VHH. A process of making monomeric polypeptides comprising single variable domain(s), e.g. Nanobody such as a VHH, a humanized VHH, an affinity-matured, stabilized or otherwise altered VHH; wherein each of the steps in the making of said polypeptide does not generate more than 10%, more preferably 5%, even more preferably 4%, even more preferably 3%, even more preferably 2%, even more preferably 1%, most preferred 0.1% w/w corresponding NFD.
55. A process according to aspect 54; wherein each of the steps in said process avoids conditions favoring hydrophobic interactions.
56. A process according to aspect 54 or aspect 55 wherein said conditions favoring hydrophobic interactions is a high concentration of said polypeptides, i.e. a concentration of said polypeptides e.g. more than 10 mg polypeptide per ml resin column material; and thus a process avoiding said interactions is avoiding such conditions in each step of its making.
57. A process according to aspect 56, wherein column loads, e.g. of an affinity column, are carefully evaluated and overload of the column is avoided, i.e. a column load maximum should be determined wherein not more than 10%, more preferably 5%, even more preferably 4%, even more preferably 3%, even more preferably 2%, even more preferably 1%, most preferred 0.1% w/w NFD is generated.
58. A process of making monomeric polypeptides comprising single variable domain(s), e.g. Nanobody such as a VHH, a humanized VHH, an affinity-matured, stabilized or otherwise altered VHH according to any of aspects 53 to 56 devoid of NFD or not more than 50%, more preferably 40%, even more preferably 30%, even more preferably 20%, most preferred 10% NFD; wherein each of the steps in said process avoids conditions favoring hydrophobic interactions, e.g. wherein the process does not consist of a protein A step and/or wherein said process avoids conditions wherein said single variable domain is partially unfolded. e.g. CDR3 is destabilized and/or partially unfolded by e.g. elevated temperature such as a temperature close to the melting temperature of the polypeptide or e.g. 37° C., over a prolonged time, e.g. weeks such as e.g. 4 weeks.
59. A pharmaceutical formulation comprising a polypeptide susceptible to dimerize, e.g. polypeptide according to a polypeptide as described in aspects 1 to 32, e.g. a polypeptide that comprises at least one of SEQ ID NO: 1, SEQ ID NO: 2, SEQ ID NO: 3, SEQ ID NO: 4, SEQ ID NO: 5, SEQ ID NO: 6, SEQ ID NO: 9 and SEQ ID NO: 10, e.g. a polypeptide that comprises polypeptide B; and polyol.
60. The pharmaceutical formulation according to aspect 59 wherein the polyol is in a concentration of e.g. not more than 0.6M.
61. The pharmaceutical formulation according to aspect 59 or 60 wherein the polyol is sorbitol, mannitol, xylitol, ribitol, and/or erythritol.
62. The pharmaceutical formulation according to aspects 59 to 61 wherein the polyol is mannitol, and e.g. in a concentration of not more than 0.6 M mannitol.
63. The pharmaceutical formulation according to aspects 59 to 62 wherein the polypeptide comprises polypeptide B.
64. The pharmaceutical formulation according to aspects 59 to 63 additionally comprising a Non-reducing sugar such as e.g. sucrose and/or trehalose and optionally NaCl and/or amino acids.
65. The pharmaceutical formulation according to aspects 59 to 64 that is a liquid formulation.
66. The pharmaceutical formulation according to aspects 59 to 64 that is prepared in a dried form, e.g. by lyophilization.
67. The pharmaceutical formulation according to aspects 59 to 64 that is used as an injectable.
68. The pharmaceutical formulation according to aspects 59 to 64 that is used as a subcutaneous formulation.
69. A NFD, a NFD fragment, or a polypeptide comprising a single variable domain that is capable of forming (or has formed) a NFD of any previous aspects e.g. as described herein wherein the single variable domain is not VHH-R9 as described in Spinelli et al, FEBS Letters 564 (2004) 35-40.

1. A stable non-fused-dimer (NFD) obtainable by a process comprising the step of concentrating a polypeptide comprising at least one single variable domain.

2. The stable NFD according to claim 1, wherein the single variable domain is a Nanobody such as a VHH, a humanized VHH, an affinity-matured, stabilized or otherwise altered VHH or a construct thereof.

3. The stable NFD according to claim 1, wherein the single variable domain is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 9 and SEQ ID NO: 10.

4. The stable NFD according to claim 1, wherein the single variable domain is SEQ ID NO: 2.

5. The stable NFD according to claim 1, wherein the single variable domain is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 9 and SEQ ID NO: 10, and of a functional sequence that is at least 70% identical to any of SEQ ID NO: 1, SEQ ID NO: 2, SEQ ID NO: 3, SEQ ID NO: 4, SEQ ID NO: 5, SEQ ID NO: 6, SEQ ID NO: 9 and SEQ ID NO: 10.

6. The stable NFD according to claim 1, wherein the single variable domain is selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 9 and SEQ ID NO: 10, and of a functional sequence that is at least 70% identical to any of SEQ ID NO: 1, SEQ ID NO: 2, SEQ ID NO: 3, SEQ ID NO: 4, SEQ ID NO: 5, SEQ ID NO: 6, SEQ ID NO: 9 and SEQ ID NO: 10; and wherein said sequences specifically bind at least to one of their target molecules.

7. The stable NFD according to claim 1, wherein the polypeptide consists essentially of a single variable domain, single variable domains, and a linker or linkers.

8. The stable NFD according to claim 1, wherein the step of concentration is done on a Protein A column and wherein high amounts of polypeptide are loaded on the column, e.g. 2 to 5 mg per ml resin Protein A.

9. The stable NFD according to claim 1, wherein the dissociation constant for the NFD to its target molecule is about the same as the dissociation constant for its corresponding monomeric building block to said target molecule.

10. The stable NFD according to claim 1, wherein there is no specific binding to its target molecule.

11. The stable NFD according to claim 1, wherein the dissociation constant for the NFD to its target molecule is 100 nM or less.

12. A polypeptide comprising at least one single variable domain; wherein said at least one of the single variable domains can form a non-fused-dimer (NFD) as described in claim 1.

13. A process of making a non-fused-dimer (NFD) according to claim 1 comprising the process step that has a condition that favors hydrophobic interactions.

14. A process of making monomeric polypeptides of the polypeptides as described in claim 1 comprising at least one single variable domain, e.g. a Nanobody; wherein each of the steps in the making of said polypeptide does not generate more than 50%, preferably 40%, more preferably 30%, more preferably 20%, even more preferably 10% corresponding non-fused-dimer (NFD); and wherein each of the steps in said process avoids conditions favoring hydrophobic interactions and/or wherein said process avoids conditions wherein said single variable domain is partially unfolded, e.g. CDR3 is partly unfolded by e.g. elevated temperature such as a temperature close to the melting temperature of the polypeptide or e.g. at 37° C., over a prolonged time, e.g. weeks such as e.g. 4 weeks.

15. A pharmaceutical formulation comprising i) a polypeptide that comprises a Nanobody that is susceptible to dimerize; and ii) a polyol.
